SEQUENCE LISTING (10 sequences)

SEQ ID NO: 1 - protein, 14 aa, Artificial Sequence
  synthetic
  MISC_FEATURE (2)..(2): Xaa represents Y, M, F or V
  MISC_FEATURE (3)..(3): Xaa represents N or G
  MISC_FEATURE (4)..(4): Xaa represents E or Q
CXXXFGCEDFYDIC

SEQ ID NO: 2 - protein, 14 aa, Artificial Sequence
  synthetic
  MISC_FEATURE (2)..(2): Xaa represents Y, M or F
  MISC_FEATURE (3)..(3): Xaa represents N or G
  MISC_FEATURE (4)..(4): Xaa represents E or Q
CXXXFGCEDFYDIC

SEQ ID NO: 3 - protein, 14 aa, Artificial Sequence
  synthetic
  MISC_FEATURE (2)..(2): Xaa represents Y or M
  MISC_FEATURE (4)..(4): Xaa represents E or Q
CXNXFGCEDFYDIC

SEQ ID NO: 4 - protein, 14 aa, Artificial Sequence
  synthetic
CYNEFGCEDFYDIC

SEQ ID NO: 5 - protein, 14 aa, Artificial Sequence
  synthetic
CMNQFGCEDFYDIC

SEQ ID NO: 6 - protein, 14 aa, Artificial Sequence
  synthetic
CFGEFGCEDFYDIC

SEQ ID NO: 7 - protein, 14 aa, Artificial Sequence
  synthetic
CVNEFGCEDFYDIC

SEQ ID NO: 8 - protein, 14 aa, Artificial Sequence
  synthetic
CFNEFGCEDFYDIC

SEQ ID NO: 9 - protein, 14 aa, Artificial Sequence
  synthetic
CYNEYGCEDFYDIC

SEQ ID NO: 10 - protein, 14 aa, Artificial Sequence
  synthetic
CYNEWGCEDFYDIC

(19) United States
(12) Patent Application Publication
(10) Pub. No.: US 2022/0135614 A1
(43) Pub. Date: May 5, 2022

(54) SYNTHESIS OF BICYCLE TOXIN CONJUGATES, AND INTERMEDIATES THEREOF

(71) Applicant: BicycleRD Limited, Cambridge (GB)

(72) Inventor: Daniel TEUFEL, Cambridge (GB)

(21) Appl. No.: 17/310,781

(22) PCT Filed: Mar. 3, 2020
(86) PCT No.: PCT/GB2020/050505

§ 371 Date: Aug. 24, 2021

Related U.S. Application Data
(60) Provisional application No. 62/813,332, filed on Mar. 4, 2019.

(51) Int. Cl. C07K 1/10; C12N 9/64; C07K 7/08; C07K 1/30

(52) U.S. Cl. CPC C07K 1/10; C12N 9/6491; C12Y 304/2408; C07K 1/30; C07K 7/08

(57) ABSTRACT

The present invention relates to Bicycle toxin conjugates, methods for preparation, and methods of use for treating cancer.

### FIELD OF THE INVENTION

The present invention relates to methods for synthesizing Bicycle toxin conjugates (BTCs), for example, BT1718, comprising a constrained bicyclic peptide covalently linked to the potent anti-tubulin agent DM1, and intermediates thereof.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å2; Wu et al. (2007), Science 330, 1066-71), a cyclic peptide with the Arg-Gly-Asp motif binding to integrin αVb3 (355 Å2) (Xiong et al. (2002), Science 296 (5565), 151-5) or the cyclic peptide inhibitor upain-1 binding to urokinase-type plasminogen activator (603 Å2; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, NIP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO2009/098450). Briefly, combinatorial libraries of linear peptides containing three cysteine residues and two regions of six random amino acids (Cys-(Xaa)6-Cys-(Xaa)6-Cys) were displayed on phage and cyclised by covalently linking the cysteine side chains to a small molecule (tris-(bromomethyl)benzene).

### SUMMARY OF THE INVENTION

The present invention provides Bicycle toxin conjugates, and methods of preparation. In some embodiments, a Bicycle toxin conjugate of the invention comprises a constrained bicyclic peptide covalently linked to the potent anti-tubulin agent DM1. In some embodiments, a Bicycle toxin conjugate comprises a constrained bicyclic peptide that binds with high affinity and specificity to membrane type 1-matrix metalloprotease (MT1-MMP).

In some embodiments, the present invention provides a Bicycle toxin conjugate of formula (I):

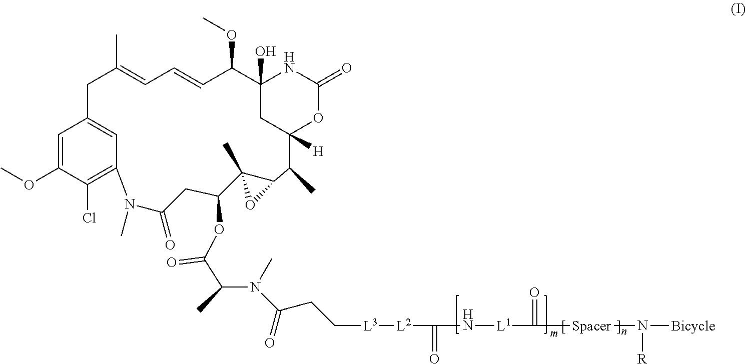

or a pharmaceutically acceptable salt thereof, wherein each of Bicycle, L¹, L², L³, Spacer, m, n, and R is as defined below and described in embodiments herein, both singly and in combination.

In some embodiments, the present invention provides a method for preparing a Bicycle toxin conjugate of the invention, or a synthetic intermediate thereof, according to schemes and steps as described herein.

In some embodiments, the present invention provides a method for preventing and/or treating cancers as described herein comprising administering to a patient a Bicycle toxin conjugate of the invention.

In some embodiments, the present invention provides a synthetic intermediate, or a composition thereof, useful for preparing a Bicycle toxin conjugate of the invention.

### DETAILED DESCRIPTION OF THE INVENTION

### 1. General Description of Certain Aspects of the Invention

A number of Bicycle toxin conjugates, and the methods of synthesis thereof, are described in International Patent Application No. PCT/GB2015/053247 (International Publication No. WO 2016/067035), the entirety of which is incorporated herein by reference. For example, a Bicycle toxin conjugate BT1718 is described as synthesized by: step 1) reacting a constrained bicyclic peptide 17-69-07-N241 with SPP (N-succinimidyl 4-(2-pyridyldithio)pentanoate) in DMSO to form an intermediate 17-69-07-N277, followed by a reverse phase purification and lyophilization to obtain pure intermediate 17-69-07-N277; and step 2) reacting the pure intermediate 17-69-07-N277 with DM1 to form BT1718 followed by standard reverse phase purification using a C18 semi-preparative column and lyophilization to obtain pure Bicycle toxin conjugate BT1718.

It has now been found that both 17-69-07-N277 and BT1718 had good solubility in polar solvents like DMF and DMA but are not soluble in non-polar solvent (e.g., MTBE), and that 17-69-07-N277 and BT1718 can be separated from impurities by precipitation in non-polar solvent (e.g., MTBE). Example 1 describes an example of an improved process for BT1718, wherein the pure intermediate in step 1) was obtained as a white powder with more than 94% purity by precipitation in MTBE, and a crude Bicycle toxin conjugate in step 2) was obtained as a white solid with more than 82% purity by precipitation in MTBE.

The improved process includes, but is not limited to, the following features:
The concentrations of the two steps were tripled, which gave a higher batch throughput (×3) of BT1718;
DMA was the only solvent utilized for the two reactions, which reduced analytical burden of testing for residual solvents;
The step 1 product N277 was isolated as a white solid by precipitation with cold MTBE (−20° C.);
The step 2 product BT1718 was isolated as a crude solid by precipitation with cold MTBE (−20° C.);
The crude product was purified by the first RP-18 column chromatography eluting with 10-45% ACN in water with 0.1% TFA. The new procedure gave very reproducible results (columns A, B and C), which shows potential to scale up to hundreds of grams. The old procedure utilized prep HPLC to purify the API, which has inherent limitation;
TFA in the combined fractions was readily purged by the second high-loading RP-18 plug eluting with 10% ACN in water. The product BT1718 was collected in high concentration when 50% ACN in water was used as an eluent;
The product solution was further concentrated and frozen in a freezer; and
BT1718 was obtained as a white fluffy solid after the lyophilization.

Accordingly, in one aspect, the present invention provides a Bicycle toxin conjugate of formula (I):

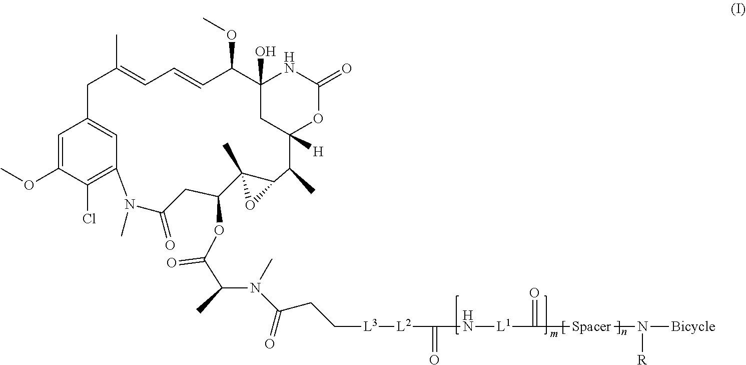

or a pharmaceutically acceptable salt thereof,
wherein:
Bicycle is a constrained bicyclic peptide that binds with high affinity and specificity to membrane type 1-matrix metalloprotease (MT1-MMP);
R is hydrogen or C₁₋₄ aliphatic;
Spacer is a natural or unnatural amino acid wherein the acid is connected to the N-terminus of Bicycle via an amide bond, or a peptide wherein the C-terminal acid of the peptide is connected to the N-terminus of Bicycle via an amide bond;
L¹ is a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—;
each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, 10-12 membered saturated or partially unsaturated bicyclic heterocyclylene having 1-4 heteroatoms independently selected from nitrogen, oxygen, or sulfur, 10-12 membered saturated or partially unsaturated tricyclic heterocyclylene having 1-4 heteroatoms independently selected from nitrogen, oxygen, or sulfur, 10-12 membered partially saturated bicyclic heteroarylene having 1-4 heteroatoms independently selected from nitrogen, oxygen, or sulfur, 10-12 membered partially saturated tricyclic heteroarylene having 1-4 heteroatoms independently selected from nitrogen, oxygen, or sulfur, 9-12 membered bicyclic heteroarylene having 1-4 heteroatoms independently selected from nitrogen, oxygen, or sulfur, 19-20 membered partially unsaturated tetracyclic heteroarylene having 1-4 heteroatoms independently selected from nitrogen, oxygen, or sulfur, or 5-6 membered heteroarylene having 1-4 heteroatoms independently selected from nitrogen, oxygen, or sulfur;
m is 0 or 1;
n is 0 or 1;
L² is a covalent bond or a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—; and
L³ is a group formed between a sulfhydryl group and a SC (sulfhydryl crosslinking) moiety.

In another aspect, the present invention provides a method for preparing a Bicycle toxin conjugate of formula (I), or a salt thereof, according to Scheme I, wherein each of the variables, reagents, intermediates, and reaction steps is as defined below and described in embodiments herein, both singly and in combination.

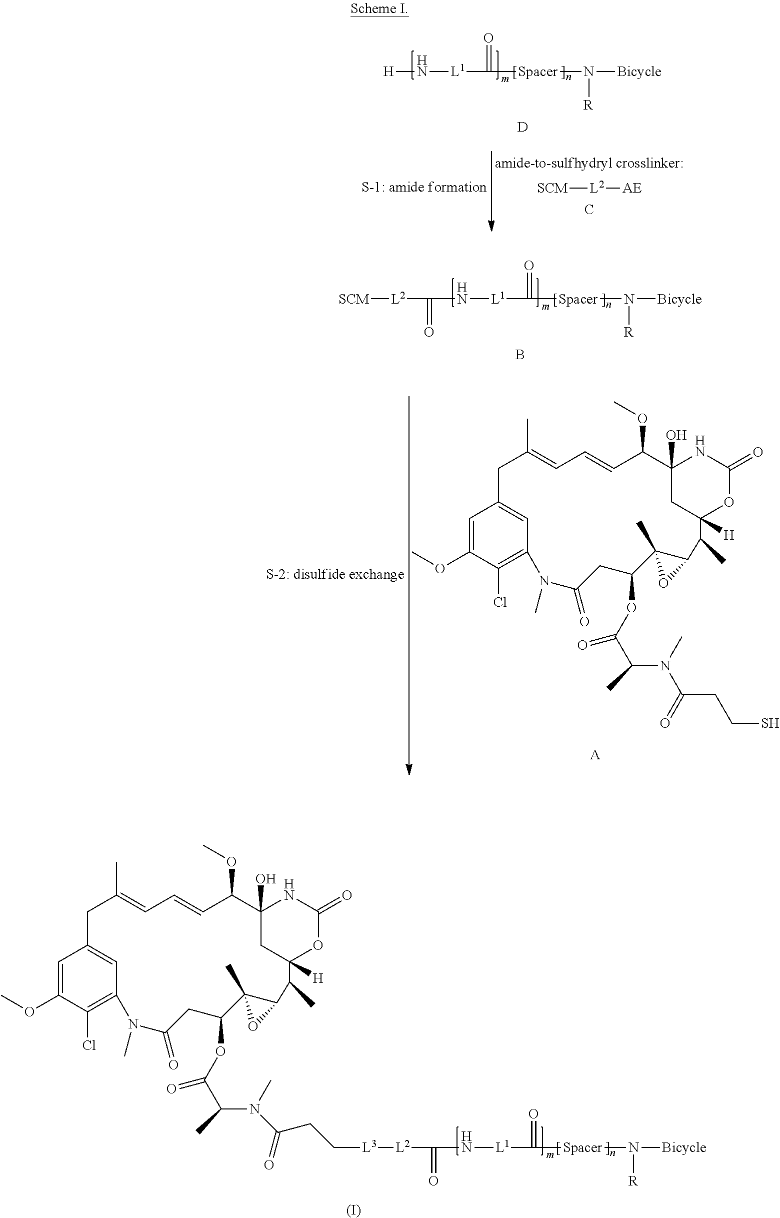

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of each of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

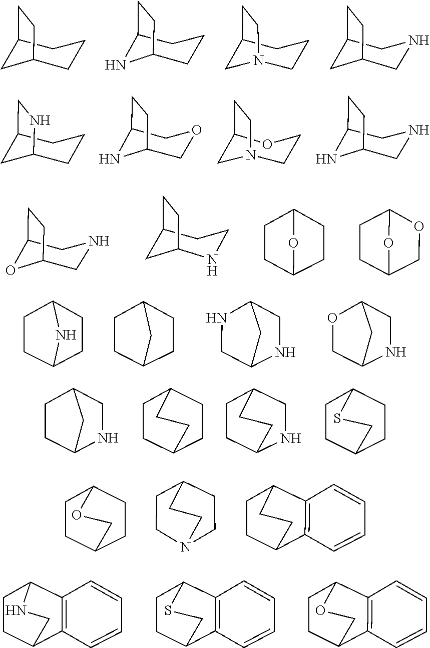

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl)).

The term “unsaturated,” as used herein, means that a moiety has one or more units of unsaturation.

As used herein, the term “bivalent hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

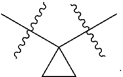 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —N(R^{∘})C(NR^{∘})N(R^{∘})₂; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{o}₃; —(CH₂)₀₋₄OC(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂0R^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{∘}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, (C₁₋₆ alkyl)sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, condition, or disorder, to treat, diagnose, prevent, and/or delay the onset of the disease, condition, or disorder. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, condition, or disorder is the amount that alleviates, ameliorates, relieves, inhibits, prevents, delays onset of, reduces severity of and/or reduces incidence of one or more symptoms or features of the disease, condition, or disorder.

The terms “treat” or “treating,” as used herein, refers to partially or completely alleviating, inhibiting, delaying onset of, preventing, ameliorating and/or relieving a disease or disorder, or one or more symptoms of the disease or disorder. As used herein, the terms “treatment,” “treat,” and “treating” refer to partially or completely alleviating, inhibiting, delaying onset of, preventing, ameliorating and/or relieving a disease or disorder, or one or more symptoms of the disease or disorder, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

Bicycle toxin conjugate BT1718 has the structure shown below, and a preparation of BT1718 is described in WO 2016/067035, the entirety of which is hereby incorporated herein by reference.

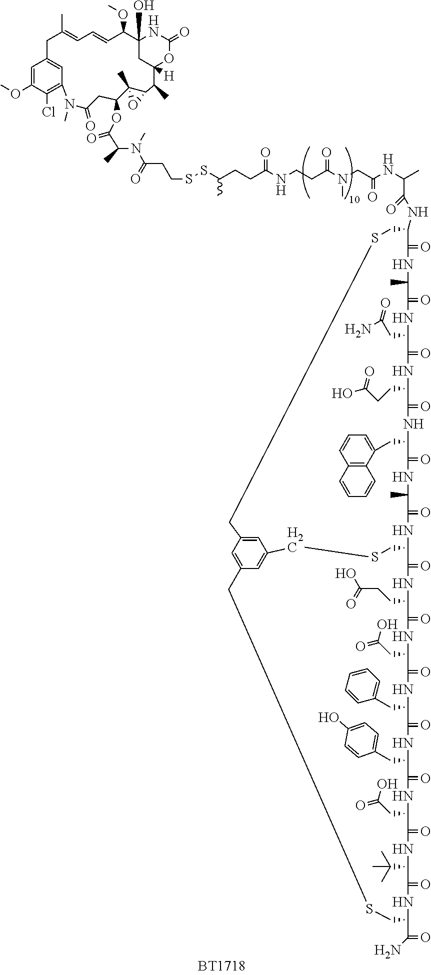

### 3. Description of Synthesis of Bicycle Toxin Conjugate of Formula (I) and Relevant Intermediates

In some embodiments, the present invention provides a method for preparing a Bicycle toxin conjugate of formula (I) according to Scheme I, wherein each of the variables, reagents, intermediates, and reaction steps is as defined below and described in embodiments herein, both singly and in combination.

Bicycle in Scheme I is a constrained bicyclic peptide that binds with high affinity and specificity to membrane type 1-matrix metalloprotease (MT1-MMP). In some embodiments, Bicycle is selected from those described in International Patent Application No. PCT/GB2015/053247 (International Publication No. WO 2016/067035), the entirety of which is incorporated herein by reference. In some embodiments, Bicycle is a peptide covalently bound to a molecular scaffold. In some embodiments, Bicycle comprises a peptide having three cysteine residues (referred as Cᵢ, Cᵢᵢ, and Cᵢᵢᵢ in the sequences below), which are capable of forming covalent bonds to a molecular scaffold. In some embodiments, Bicycle comprises a peptide -Cᵢ-Y/M/F/V-N/G-E/Q-F-G-Cᵢᵢ-E-D-F-Y-D-I-Cᵢᵢᵢ- (SEQ ID NO: 1). In some embodiments, Bicycle comprises a peptide -Cᵢ-Y/M/F-N/G-E/Q-F-G-Cᵢᵢ-E-D-F-Y-D-I-Cᵢᵢᵢ- (SEQ ID NO: 2). In some embodiments, Bicycle comprises a peptide -Cᵢ-Y/M-N-E/Q-F-G-Cᵢᵢ-E-D-F-Y-D-I-Cᵢᵢᵢ- (SEQ ID NO: 3). In some embodiments Bicycle comprises a peptide selected from:

(SEQ ID NO: 4)
-Cᵢ-Y-N-E-F-G-Cᵢᵢ-E-D-F-Y-D-I-Cᵢᵢᵢ-;
(SEQ ID NO: 5)
-Cᵢ-M-N-Q-F-G-Cᵢᵢ-E-D-F-Y-D-I-Cᵢᵢᵢ-;
(SEQ ID NO: 6)
-Cᵢ-F-G-E-F-G-Cᵢᵢ-E-D-F-Y-D-I-Cᵢᵢᵢ-;
(SEQ ID NO: 7)
-Cᵢ-V-N-E-F-G-Cᵢᵢ-E-D-F-Y-D-I-Cᵢᵢᵢ-;
(SEQ ID NO: 8)
-Cᵢ-F-N-E-F-G-Cᵢᵢ-E-D-F-Y-D-I-Cᵢᵢᵢ-;
(SEQ ID NO: 9)
-Cᵢ-Y-N-E-Y-G-Cᵢᵢ-E-D-F-Y-D-I-Cᵢᵢᵢ-;
and
(SEQ ID NO: 10)
-Cᵢ-Y-N-E-W-G-Cᵢᵢ-E-D-F-Y-D-I-Cᵢᵢᵢ-;

In some embodiments, Bicycle is:

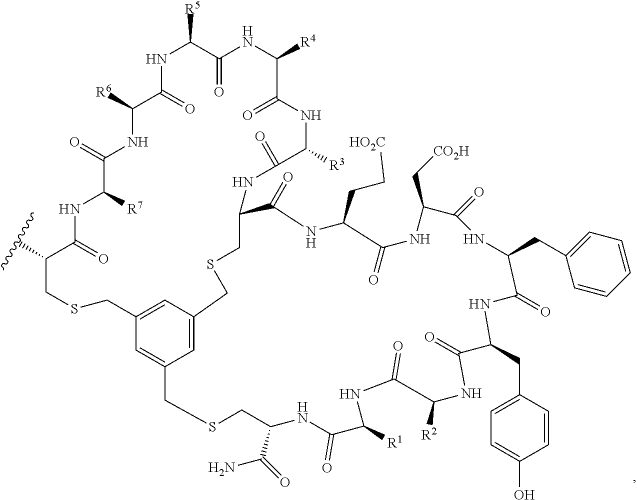

wherein each of R¹, R², R³, R⁴, R⁵, R⁶, and R⁷ is as independently defined below and described in embodiments herein, both singly and in combination.

In some embodiments, each of R¹, R², R³, R⁴, R⁵, R⁶, and R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In certain embodiments, R¹ is hydrogen or C₁₋₆ aliphatic. In certain embodiments, R¹ is t-butyl.

In certain embodiments, R² is hydrogen or optionally substituted C₁₋₆ aliphatic. In certain embodiments, R² is

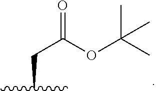

In certain embodiments, R³ is hydrogen or C₁₋₆ aliphatic. In certain embodiments, R³ is methyl.

In certain embodiments, R⁴ is hydrogen or optionally substituted C₁₋₆ aliphatic. In certain embodiments, R⁴ is

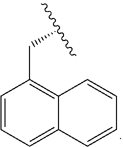

In certain embodiments, R⁵ is hydrogen or optionally substituted C₁₋₆ aliphatic. In certain embodiments, R⁵ is

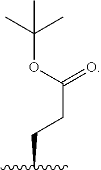

In certain embodiments, R⁶ is hydrogen or optionally substituted C₁₋₆ aliphatic. In certain embodiments, R⁶ is

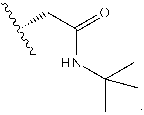

In certain embodiments, R⁷ is hydrogen or C₁₋₆ aliphatic. In certain embodiments, R⁷ is methyl.

The R group in Scheme I is hydrogen or C₁₋₄ aliphatic. In some embodiments, R is H. In some embodiments, R is C₁₋₄ aliphatic. In some embodiments, R is C₁₋₄ alkyl. In some embodiments, R is methyl. In some embodiments, R is ethyl. In some embodiments, R is isopropyl. In some embodiments, R is propyl. In some embodiments, R is butyl. In some embodiments, R is isobutyl. In some embodiments, R is t-butyl.

The Spacer moiety in Scheme I is a natural or unnatural amino acid wherein the acid is connected to the N-terminus of Bicycle via an amide bond, or a peptide wherein the C-terminal acid of the peptide is connected to the N-terminus of Bicycle via an amide bond. In some embodiments, Spacer is a natural amino acid wherein the acid is connected to the N-terminus of Bicycle via an amide bond. In some embodiments, Spacer is an unnatural amino acid wherein the acid is connected to the N-terminus of Bicycle via an amide bond. In some embodiments, Spacer is a peptide wherein the C-terminal acid of the peptide is connected to the N-terminus of Bicycle via an amide bond.

In some embodiments, Spacer is L-Alanine. In some embodiments, Spacer is D-Alanine.

In some embodiments, Spacer is

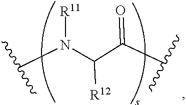

wherein:
each of R¹¹ is independently hydrogen or C₁₋₄ aliphatic;
each of R¹² is independently hydrogen, or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or
an R² group and its adjacent R¹¹ group are optionally taken together with their intervening atoms to form a 4-8 membered saturated or partially unsaturated monocyclic heterocyclic ring having 1-2 heteroatoms independently selected from nitrogen, oxygen, or sulfur; and
s is 1-12.

In some embodiments, Spacer is

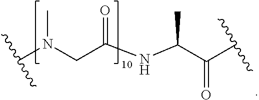

The L¹ group in Scheme I is a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—, wherein each of -Cy¹- and R is independently as defined and described in embodiments herein, both singly and in combination.

In some embodiments, L¹ is a C₁₋₂₀ bivalent hydrocarbon chain wherein 1-3 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—, wherein each of -Cy¹- and R is independently as defined and described in embodiments herein, both singly and in combination. In some embodiments, L¹ is a C₁₋₁₂ bivalent hydrocarbon chain wherein 1 or 2 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, or —(CH₂CH₂O)₁₋₂₀—, wherein each of -Cy¹- and R is independently as defined and described in embodiments herein, both singly and in combination. In some embodiments, L¹ is a C₁₋₂₀ bivalent hydrocarbon chain wherein 1-3 methylene units of the chain are optionally and independently replaced by —(CH₂CH₂O)₁₋₂₀—.

In some embodiments, L¹ is an unsubstituted C₁₋₂₀ bivalent hydrocarbon chain. In some embodiments, L¹ is an unsubstituted C₁₋₁₂ bivalent hydrocarbon chain. In some embodiments, L¹ is an unsubstituted C₁₋₆ bivalent hydrocarbon chain. In some embodiments, L¹ is —CH₂CH₂—.

Each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, 10-12 membered saturated or partially unsaturated bicyclic heterocyclylene having 1-4 heteroatoms independently selected from nitrogen, oxygen, or sulfur, 10-12 membered saturated or partially unsaturated tricyclic heterocyclylene having 1-4 heteroatoms independently selected from nitrogen, oxygen, or sulfur, 10-12 membered partially saturated bicyclic heteroarylene having 1-4 heteroatoms independently selected from nitrogen, oxygen, or sulfur, 10-12 membered partially saturated tricyclic heteroarylene having 1-4 heteroatoms independently selected from nitrogen, oxygen, or sulfur, 9-12 membered bicyclic heteroarylene having 1-4 heteroatoms independently selected from nitrogen, oxygen, or sulfur, 19-20 membered partially unsaturated tetracyclic heteroarylene having 1-4 heteroatoms independently selected from nitrogen, oxygen, or sulfur, and 5-6 membered heteroarylene having 1-4 heteroatoms independently selected from nitrogen, oxygen, or sulfur. In some embodiments, each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, and 5-6 membered heteroarylene having 1-4 heteroatoms independently selected from nitrogen, oxygen, or sulfur.

Each of m and n in Scheme I is independently 0 or 1. In some embodiments, m is 0. In some embodiments, m is 1. In some embodiments, n is 0. In some embodiments, n is 1.

Compound C in Scheme I is an amide-to-sulfhydryl crosslinker, which is used for conjugation between a primary amine group and a sulfhydryl group. Compound C comprises an active ester moiety (AEM) and a sulfhydryl crosslinking moiety (SCM), which are connected by a L² group. In some embodiments, compound C is an NHS-Haloacetyl crosslinker. In some embodiments, compound C is SIA (succinimidyl iodoacetate):

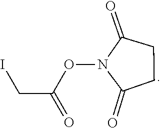 [SMILES: O=C(CI)ON1C(=O)CCC1=O]

In some embodiments, compound C is SBAP (succinimidyl 3-(bromoacetamido)propionate):

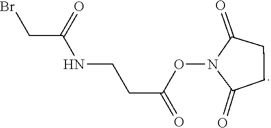 [SMILES: O=C(CBr)NCCC(=O)ON1C(=O)CCC1=O]

In some embodiments, compound C is SIAB (succinimidyl (4-iodoacetyl)aminobenzoate):

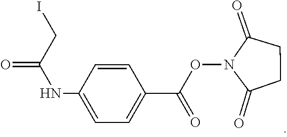 [SMILES: O=C(CI)Nc1ccc(C(=O)ON2C(=O)CCC2=O)cc1]

In some embodiments, compound C is Sulfo-SIAB (sulfosuccinimidyl (4-iodoacetyl)aminobenzoate):

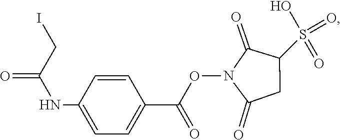 [SMILES: O=C(CI)Nc1ccc(C(=O)ON2C(=O)CC(S(=O)(=O)O)C2=O)cc1]

or a salt (e.g., a sodium salt) thereof. In some embodiments, compound C is an NHS-Maleimide crosslinker. In some embodiments, compound C is AMAS (N-α-maleimidoacet-oxysuccinimide ester):

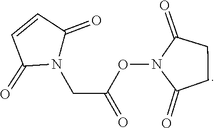 [SMILES: O=C(CN1C(=O)C=CC1=O)ON1C(=O)CCC1=O]

In some embodiments, compound C is BMPS (N-β-maleimidopropyl-oxysuccinimide ester):

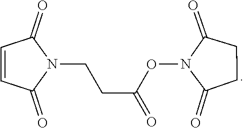 [SMILES: O=C(CCN1C(=O)C=CC1=O)ON1C(=O)CCC1=O]

In some embodiments, compound C is GMBS (N-γ-maleimidobutyryl-oxysuccinimide ester):

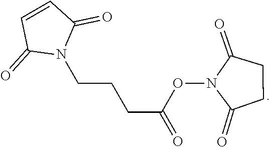 [SMILES: O=C(CCCN1C(=O)C=CC1=O)ON1C(=O)CCC1=O]

In some embodiments, compound C is Sulfo-GMBS (N-γ-maleimidobutyryl-oxysulfosuccinimide ester):

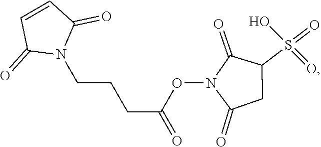 [SMILES: O=C(CCCN1C(=O)C=CC1=O)ON1C(=O)CC(S(=O)(=O)O)C1=O]

or a salt (e.g., a sodium salt) thereof. In some embodiments, compound C is MBS (m-maleimidobenzoyl-N-hydroxysuccinimide ester):

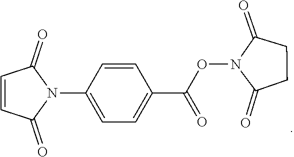 [SMILES: O=C(ON1C(=O)CCC1=O)c1ccc(N2C(=O)C=CC2=O)cc1]

In some embodiments, compound C is Sulfo-MBS (m-maleimidobenzoyl-N-hydroxysulfosuccinimide ester):

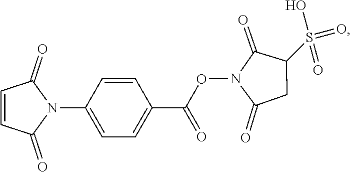 [SMILES: O=C(ON1C(=O)CC(S(=O)(=O)O)C1=O)c1ccc(N2C(=O)C=CC2=O)cc1]

or a salt (e.g., a sodium salt) thereof. In some embodiments, compound C is SMCC (succinimidyl 4-(N-maleimidomethyl)cyclohexane-1-carboxylate):

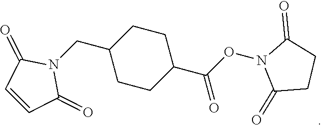 [SMILES: O=C(ON1C(=O)CCC1=O)C1CCC(CN2C(=O)C=CC2=O)CC1]

In some embodiments, compound C is Sulfo-SMCC (sulfosuccinimidyl 4-(N-maleimidomethyl)cyclohexane-1-carboxylate):

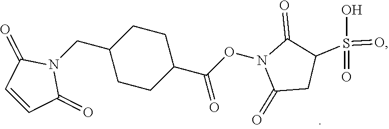 [SMILES: O=C(ON1C(=O)CC(S(=O)(=O)O)C1=O)C1CCC(CN2C(=O)C=CC2=O)CC1]

or a salt (e.g., a sodium salt) thereof. In some embodiments, compound C is EMCS (N-ε-malemidocaproyl-oxysuccinimide ester):

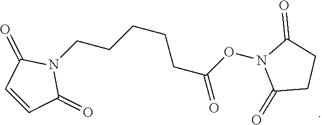 [SMILES: O=C(CCCCCN1C(=O)C=CC1=O)ON1C(=O)CCC1=O]

In some embodiments, compound C is Sulfo-EMCS (N-ε-maleimidocaproyl-oxysulfosuccinimide ester):

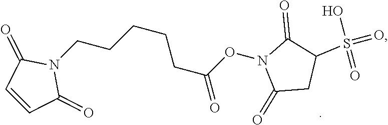 [SMILES: O=C(CCCCCN1C(=O)C=CC1=O)ON1C(=O)CC(S(=O)(=O)O)C1=O]

or a salt (e.g., a sodium salt) thereof. In some embodiments, compound C is SMPB (succinimidyl 4-(p-maleimidophenyl)butyrate):

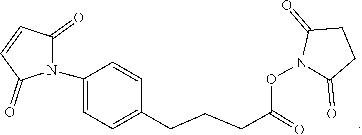 [SMILES: O=C(CCCc1ccc(N2C(=O)C=CC2=O)cc1)ON1C(=O)CCC1=O]

In some embodiments, compound C is Sulfo-SMPB (sulfosuccinimidyl 4-(N-maleimidophenyl)butyrate):

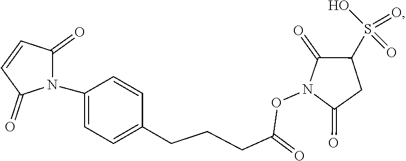 [SMILES: O=C(CCCc1ccc(N2C(=O)C=CC2=O)cc1)ON1C(=O)CC(S(=O)(=O)O)C1=O]

or a salt (e.g., a sodium salt) thereof. In some embodiments, compound C is SMPH (Succinimidyl 6-((beta-maleimidopropionamido)hexanoate)):

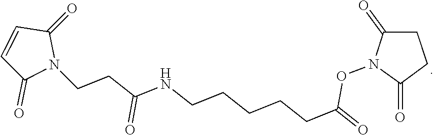 [SMILES: O=C(CCN1C(=O)C=CC1=O)NCCCCCC(=O)ON1C(=O)CCC1=O]

In some embodiments, compound C is LC-SMCC (succinimidyl 4-(N-maleimidomethyl)cyclohexane-1-carboxy-(6-amidocaproate)):

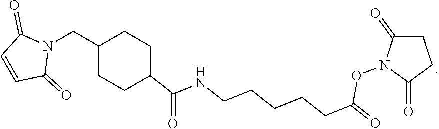 [SMILES: O=C(CCCCCNC(=O)C1CCC(CN2C(=O)C=CC2=O)CC1)ON1C(=O)CCC1=O]

In some embodiments, compound C is Sulfo-KMUS (N-κ-maleimidoundecanoyl-oxysulfosuccinimide ester):

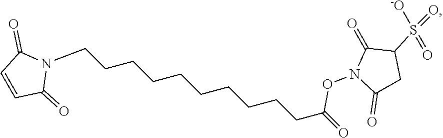

or a salt (e.g., a sodium salt) thereof. In some embodiments, compound C is an NHS-Pyridyldithiol crosslinker. In some embodiments, compound C is SPDP (succinimidyl 3-(2-pyridyldithio)propionate):

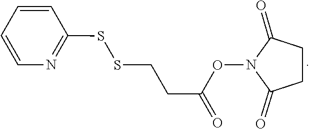 [SMILES: O=C(CCSSc1ccccn1)ON1C(=O)CCC1=O]

In some embodiments, compound C is LC-SPDP:

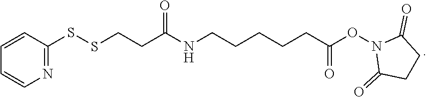 [SMILES: O=C(CCSSc1ccccn1)NCCCCCC(=O)ON1C(=O)CCC1=O]

In some embodiments, compound C is Sulfo-LC-SPDP (sulfosuccinimidyl 6-(3′-(2-pyridyldithio)propionamido)hexanoate):

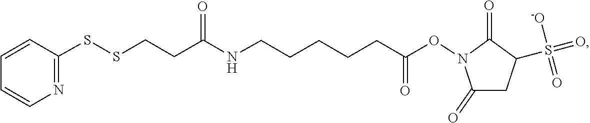

or a salt (e.g., a sodium salt) thereof. In some embodiments, compound C is SMPT (4-succinimidyloxycarbonyl-alpha-methyl-α(2-pyridyldithio)toluene):

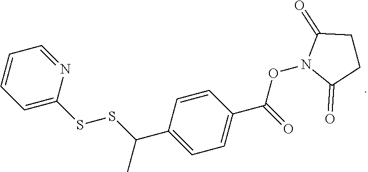 [SMILES: CC(SSc1ccccn1)c1ccc(C(=O)ON2C(=O)CCC2=O)cc1]

In some embodiments, compound C is PEG4-SPDP (PEGylated, long-chain SPDP crosslinker):

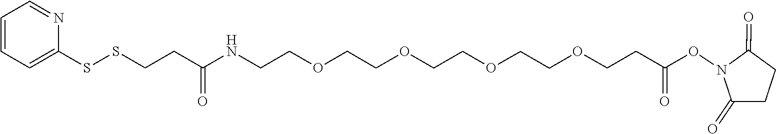 [SMILES: O=C(CCSSc1ccccn1)NCCOCCOCCOCCOCCC(=O)ON1C(=O)CCC1=O]

In some embodiments, compound C is PEG12-SPDP (PEGylated, long-chain SPDP crosslinker):

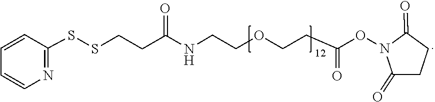

In some embodiments, compound C is

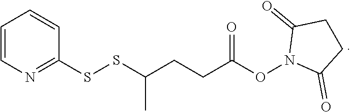 [SMILES: CC(CCC(=O)ON1C(=O)CCC1=O)SSc1ccccn1]

The active ester moiety (AEM) of compound C is

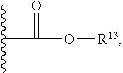

wherein —O—R¹³ is a leaving group. In some embodiments, R¹³ is

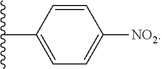 [SMILES: *c1ccc([N+](=O)[O-])cc1]

In some embodiments, R¹³ is

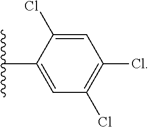 [SMILES: *c1cc(Cl)c(Cl)cc1Cl]

In some embodiments, R¹³ is

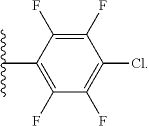 [SMILES: *c1c(F)c(F)c(Cl)c(F)c1F]

In some embodiments, R¹³ is

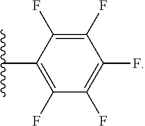 [SMILES: *c1c(F)c(F)c(F)c(F)c1F]

In some embodiments, R¹³ is

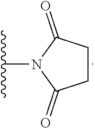 [SMILES: *N1C(=O)CCC1=O]

In some embodiments, R¹³ is

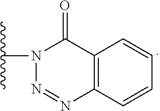 [SMILES: *n1nnc2ccccc2c1=O]

In some embodiments, R¹³ is

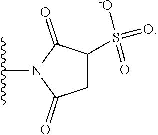

The L² group in Scheme I is a covalent bond or a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—, wherein each of -Cy¹- and R is independently as defined and described in embodiments herein, both singly and in combination.

In some embodiments, L² is a covalent bond. In some embodiments, L² is a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—, wherein each of -Cy¹- and R is independently as defined and described in embodiments herein, both singly and in combination.

In some embodiments, L² is a C₁₋₁₂ bivalent hydrocarbon chain wherein 1-3 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—, wherein each of -Cy¹- and R is independently as defined and described in embodiments herein, both singly and in combination.

In some embodiments, L² is a C₁₋₁₂ bivalent hydrocarbon chain wherein 1 or 2 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, or —(CH₂CH₂O)₁₋₂₀—, wherein each of -Cy¹- and R is independently as defined and described in embodiments herein, both singly and in combination.

In some embodiments, L² is a C₁₋₁₂ bivalent hydrocarbon chain wherein 1 or 2 methylene units of the chain is optionally replaced by

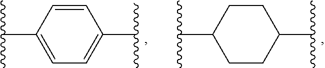

—N(R)—, —C(O)N(R)—, —N(R)C(O)—, or —(CH₂CH₂O)₁₋₂₀—.

In some embodiments, L² is

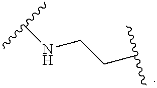 [SMILES: *CCN*]

In some embodiments, L² is

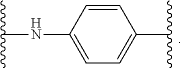 [SMILES: *Nc1ccc(*)cc1]

In some embodiments, L² is —CH₂—. In some embodiments, L² is —(CH₂)₂—. In some embodiments, L² is —(CH₂)₃—. In some embodiments, L² is

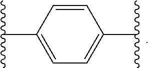 [SMILES: *c1ccc(*)cc1]

In some embodiments, L² is

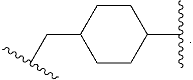 [SMILES: *CC1CCC(*)CC1]

In some embodiments, L² is —(CH₂)₅—. In some embodiments, L² is —(CH₂)₄—. In some embodiments, L² is

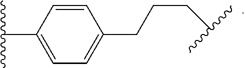 [SMILES: *CCCc1ccc(*)cc1]

In some embodiments, L² is

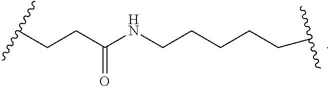 [SMILES: *CCCCCNC(=O)CC*]

In some embodiments, L² is

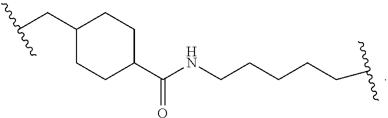 [SMILES: *CCCCCNC(=O)C1CCC(C*)CC1]

In some embodiments, L² is —(CH₂)₁₀—. In some embodiments, L² is

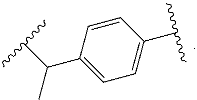 [SMILES: *c1ccc(C(*)C)cc1]

In some embodiments, L² is

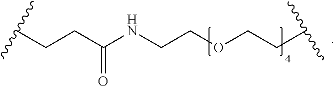

In some embodiments, L² is

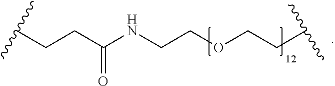

In some embodiments, L² is

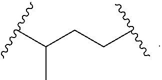 [SMILES: *CCC(*)C]

The sulfhydryl crosslinking moiety (SCM) of compounds C and B in Scheme I is a moiety that forms a bond with sulfhydryl (—SH). In some embodiments, SCM is haloacetyl. In some embodiments, SCM is

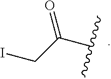 [SMILES: *C(=O)CI]

In some embodiments, SCM is Br

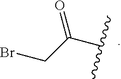 [SMILES: *C(=O)CBr]

In some embodiments, SCM is maleimide:

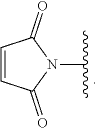 [SMILES: *N1C(=O)C=CC1=O]

In some embodiments, SCM is pyridyl disulfide. In some embodiments, SCM is

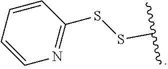 [SMILES: *SSc1ccccn1]

The L³ group in Scheme I is a group formed between a sulfhydryl group of compound A, and a sulfhydryl crosslinking moiety (SCM) of compound B. In some embodiments, L³ is

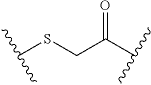 [SMILES: *SCC(*)=O]

which is formed, for example, between the sulfhydryl group of compound A, and haloacetyl of compound B (which is a sulfhydryl crosslinking moiety as described herein). In some embodiments, L³ is

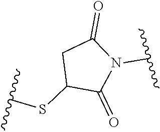 [SMILES: *SC1CC(=O)N(*)C1=O]

which is formed, for example, between the sulfhydryl group of compound A, and maleimide of compound B (which is a sulfhydryl crosslinking moiety as described herein). In some embodiments, L³ is —S—S— which is formed, for example, between the sulfhydryl group of compound A, and pyridyl disulfide of compound B (which is a sulfhydryl crosslinking moiety as described herein).

Compound D can be prepared or isolated in general by synthetic and/or semi-synthetic methods known to those skilled in the art for analogous compound (for example, as described in WO 2016/067035, the entire content of which is incorporated herein by reference) and by methods described in detail in the Examples, herein.

In some embodiments, compound D in Scheme I is:

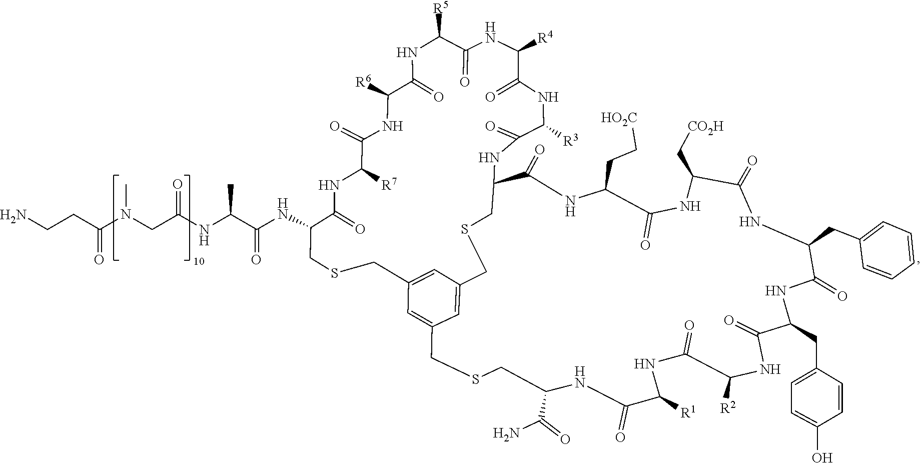

or a salt thereof, wherein each of R¹, R², R³, R⁴, R⁵, R⁶, and R⁷ is as defined below and described in embodiments herein, both singly and in combination.

In some embodiments, compound D in Scheme I is:

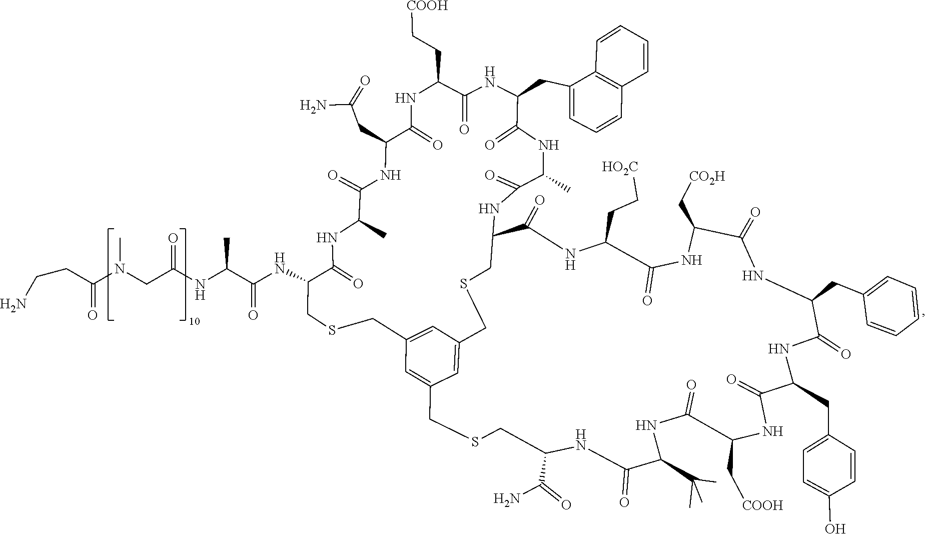

or a salt thereof.

In some embodiments, compound B in Scheme I is:

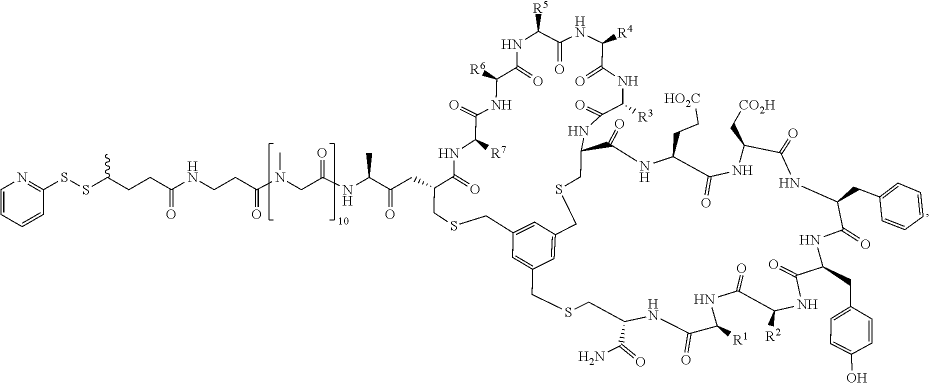

or a salt thereof, wherein each of R¹, R², R³, R⁴, R⁵, R⁶, and R⁷ is as defined below and described in embodiments herein, both singly and in combination.

In some embodiments, compound B in Scheme I is:

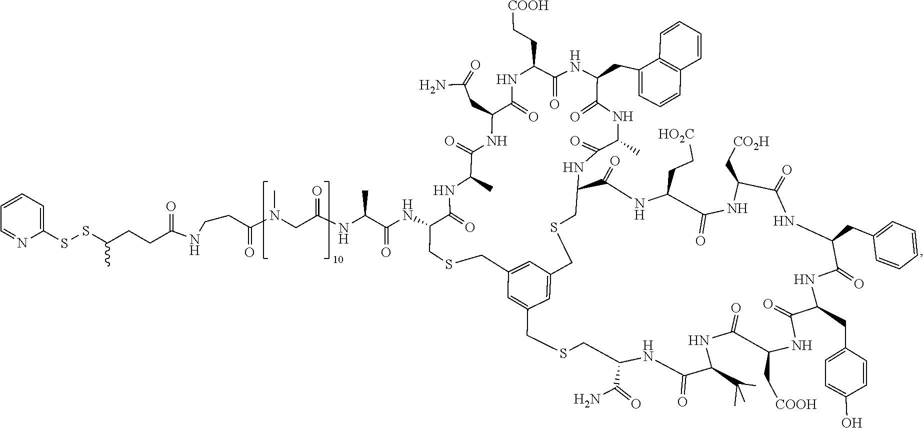

or a salt thereof.

At Step S-1 (amide formation), compound D, or a salt thereof, is coupled to compound C, or a salt thereof, to form compound B, or a salt thereof. Suitable coupling reactions are well known to one of ordinary skill in the art and typically involve an activated ester derivative such that treatment with an amine moiety results in the formation of an amide bond. The coupling reaction is typically carried out in the presence of an excess of a base. In some embodiments, the base is a tertiary amine base. In some embodiments, the tertiary amine base is triethylamine. In some embodiments, the base is a tertiary amine base. In some embodiments, the tertiary amine base is N,N-Diisopropylethylamine (DIPEA). The coupling reaction may be carried out in a suitable solvent that solubilizes all of the reagents. In some embodiments, the solvent is a dipolar aprotic solvent. In some embodiments, the dipolar aprotic solvent is N,N-dimethylacetamide (DMA). In some embodiments, the dipolar aprotic solvent is dimethyl sulfoxide (DMSO), N,N-dimethylformamide (DMF), acetone, ethyl acetate, hexamethylphosphoramide (HMPA) or N,N′-dimethylpropyleneurea (DMPU). In some embodiments, the reaction mixture is mixed with a non-polar solvent to precipitate out compound B, or a salt thereof. In some embodiments, the reaction mixture is mixed with a non-polar solvent at room temperature or a lower temperature to form a suspension or slurry. In some embodiments, the suspension or slurry is further stored at room temperature or a lower temperature for a period of time, with or without mixing, before compound B, or a salt thereof, is filtered out. In some embodiments, a lower temperature is about 15° C., 10° C., 5° C., 0° C., −5° C., −10° C., −15° C., or −20° C. In some embodiments, a lower temperature is below −20° C. In some embodiments, a non-polar solvent is an ether. In some embodiments, a non-polar solvent is diethyl ether. In some embodiments, a non-polar solvent is methyl tert-butyl ether (MTBE). In some embodiments, compound B, or a salt thereof, obtained by precipitation and filtration is of a purity of about 80% or higher. In some embodiments, compound B, or a salt thereof, obtained by precipitation and filtration is of a purity of about 82%, 84%, 86%, 88%, 90%, 92%, 94%, 96%, or 98%. In some embodiments, compound B, or a salt thereof, obtained by precipitation and filtration is further purified by column chromatography.

At Step S-2 (disulfide exchange), compound B, or a salt thereof, and compound A, or a salt thereof, have a sulfhydryl crosslinking reaction to form a compound of formula (I), or a salt thereof. Suitable crosslinking reactions are well known to one of ordinary skill in the art and typically involve a sulfhydryl crosslinking moiety such that treatment with a thiol moiety results in the formation of a disulfide bond. The coupling reaction is typically carried out in the presence of an excess of a base. In some embodiments the base is a tertiary amine base. In some embodiments, the tertiary amine base is triethylamine. In some embodiments the base is a tertiary amine base. In some embodiments, the tertiary amine base is DIPEA. The coupling reaction may be carried out in a suitable solvent that solubilizes all of the reagents. In some embodiments, the solvent is a dipolar aprotic solvent. In some embodiments, the dipolar aprotic solvent is DMA. In some embodiments, the dipolar aprotic solvent is DMSO, DMF, acetone, ethyl acetate, HMPA or DMPU. In some embodiments, the reaction mixture is mixed with a non-polar solvent to precipitate out the compound of formula (I), or a salt thereof. In some embodiments, the reaction mixture is mixed with a non-polar solvent at room temperature or a lower temperature to form a suspension or slurry. In some embodiments, the suspension or slurry is further stored at room temperature or a lower temperature for a period of time, with or without mixing, before a compound of formula (I), or a salt thereof, is filtered out. In some embodiments, a lower temperature is about 15° C., 10° C., 5° C., 0° C., −5° C., −10° C., −15° C., or −20° C. In some embodiments, a lower temperature is below −20° C. In some embodiments, a non-polar solvent is an ether. In some embodiments, a non-polar solvent is diethyl ether. In some embodiments, a non-polar solvent is methyl tert-butyl ether (MTBE). In some embodiments, a compound of formula (I), or a salt thereof, obtained by precipitation and filtration is of a purity of about 70% or higher. In some embodiments, a compound of formula (I), or a salt thereof, obtained by precipitation and filtration is of a purity of about 72%, 74%, 76%, 78%, 80%, 82%, 84%, 86%, 88%, 90%, 92%, 94%, 96%, or 98%. In some embodiments, a compound of formula (I), or a salt thereof, obtained by precipitation and filtration is further purified by column chromatography.

In some embodiments, the present invention provides a method for preparing compound B, or a salt thereof, comprising steps of 1) providing compound D, or a salt thereof; 2) reacting compound D, or a salt thereof, with compound C, or a salt thereof, to form compound B, or a salt thereof; and 3) separating compound B, or a salt thereof, from reaction mixture by precipitation, wherein each of compounds B, C, and D is as described above. In some embodiments, the method further comprises purifying compound B, or a salt thereof, by column chromatography. In some embodiments, solvents and conditions of the method are as described for step S-1 above.

In some embodiments, the present invention provides a method for preparing a compound of formula (I), or a salt thereof, comprising steps of 1) providing compound B, or a salt thereof; 2) reacting compound B, or a salt thereof, with compound A, or a salt thereof, to form a compound of formula (I), or a salt thereof; and 3) separating the compound of formula (I), or a salt thereof, from reaction mixture by precipitation, wherein each of compounds B and A, and a compound of formula (I) is as described above. In some embodiments, the method further comprises purifying the compound of formula (I), or a salt thereof, by column chromatography. In some embodiments, solvents and conditions of the method are as described for step S-2 above.

In some embodiments, the present invention provides a method for preparing a compound of formula (I), or a salt thereof, comprising steps of 1) providing compound D, or a salt thereof; 2) reacting compound D, or a salt thereof, with compound C, or a salt thereof, to form compound B, or a salt thereof; 3) separating compound B, or a salt thereof, from reaction mixture by precipitation; 4) reacting compound B, or a salt thereof, with compound A, or a salt thereof, to form a compound of formula (I), or a salt thereof; and 5) separating the compound of formula (I), or a salt thereof, from reaction mixture by precipitation. In some embodiments, the method further comprises purifying the compound of formula (I), or a salt thereof, by column chromatography. In some embodiments, compound B, or a salt thereof, obtained from step 3) is not further purified by column chromatography before being used in step 4). In some embodiments, solvents and conditions of the method are as described for steps S-1 and S-2 above.

In some embodiments, the present invention provides a heterogeneous mixture comprising compound B, or a salt thereof, and a non-polar solvent. In some embodiments, a heterogeneous mixture is a suspension. In some embodiments, a heterogeneous mixture is a slurry. In some embodiments, the present invention provides a solid composition comprising compound B, or a salt thereof, and a small amount of a non-polar solvent. In some embodiments, the heterogeneous mixture and/or solid composition further comprise N-hydroxysuccinimide. In some embodiments, the non-polar solvent in the heterogeneous mixture and/or solid composition is as described for step S-1 above. In some embodiments, the temperature of the heterogeneous mixture and/or solid composition is as described for step S-1 above. In some embodiments, purity of compound B, or a salt thereof, after being filtered out of the heterogeneous mixture is as described for step S-1 above. In some embodiments, purity of compound B, or a salt thereof, in the solid composition is as described for step S-1 above.

In some embodiments, the present invention provides a heterogeneous mixture comprising a compound of formula (I), or a salt thereof, and a non-polar solvent. In some embodiments, a heterogeneous mixture is a suspension. In some embodiments, a heterogeneous mixture is a slurry. In some embodiments, the present invention provides a solid composition comprising a compound of formula (I), or a salt thereof, and a small amount of a non-polar solvent. In some embodiments, the heterogeneous mixture and/or solid composition further comprises 2-pyridinethiol. In some embodiments, the non-polar solvent in the heterogeneous mixture and/or solid composition is as described for step S-2 above. In some embodiments, the temperature of the heterogeneous mixture and/or solid composition is as described for step S-2 above. In some embodiments, purity of compound of formula (I), or a salt thereof, after being filtered out of the heterogeneous mixture is as described for step S-2 above. In some embodiments, purity of compound of formula (I), or a salt thereof, in the solid composition is as described for step S-2 above.

### 4. Description of Exemplary Bicycle Toxin Conjugates

In some embodiments, a Bicycle toxin conjugate of formula (I) is:

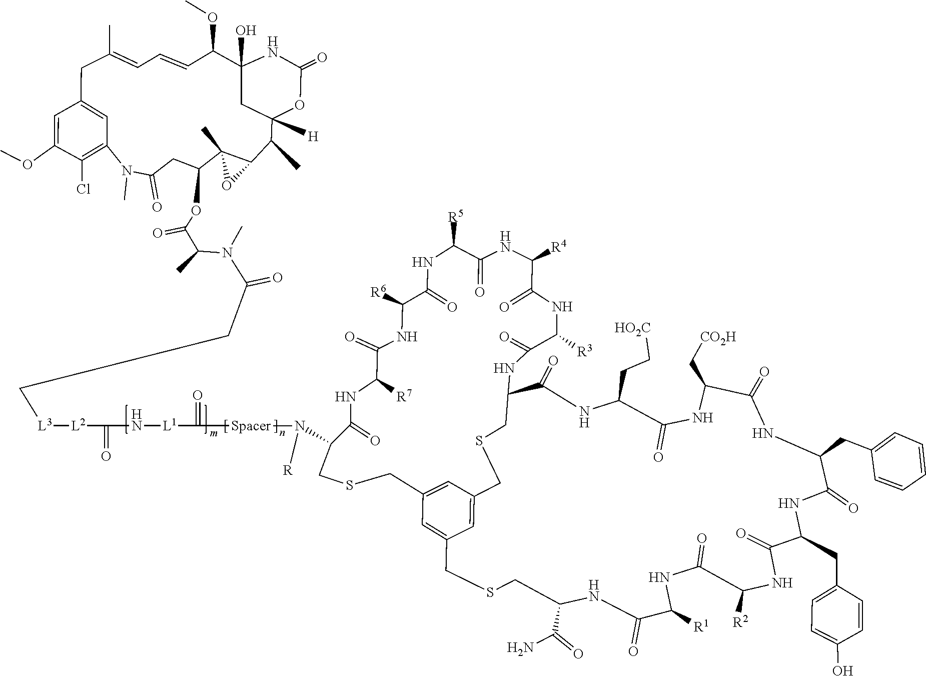

or a pharmaceutically acceptable salt thereof, wherein each of L¹, L², L³, Spacer, m, n, R, R¹, R², R³, R⁴, R⁵, R⁶, and R⁷ is as described in embodiments herein, both singly and in combination.

In some embodiments a Bicycle toxin conjugate of formula (I) is:

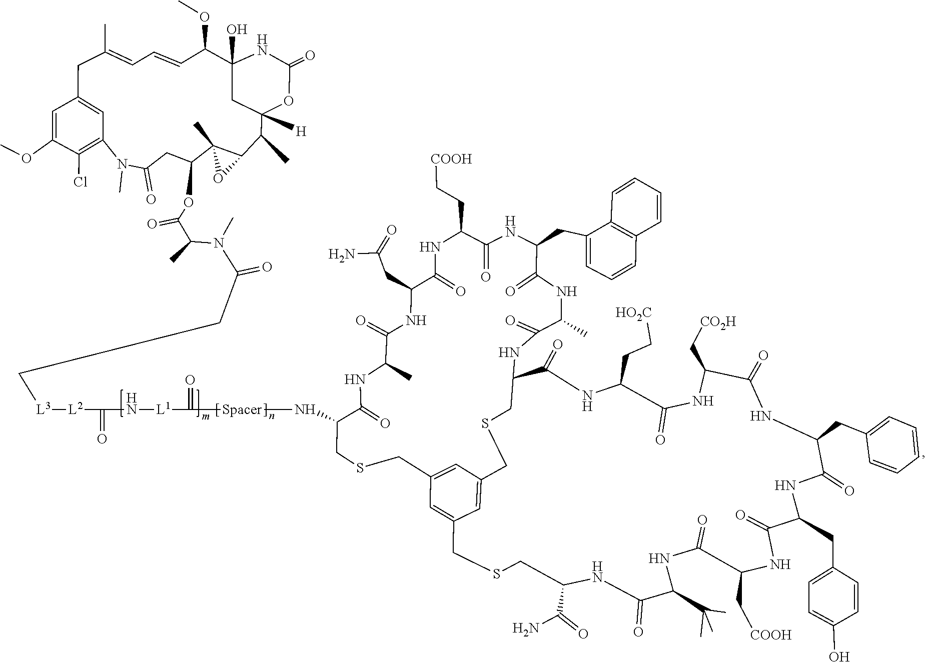

or a pharmaceutically acceptable salt thereof, wherein each of L¹, L², L³, Spacer, m, and n is as described in embodiments herein, both singly and in combination.

In some embodiments, a Bicycle toxin conjugate of formula (I) is:

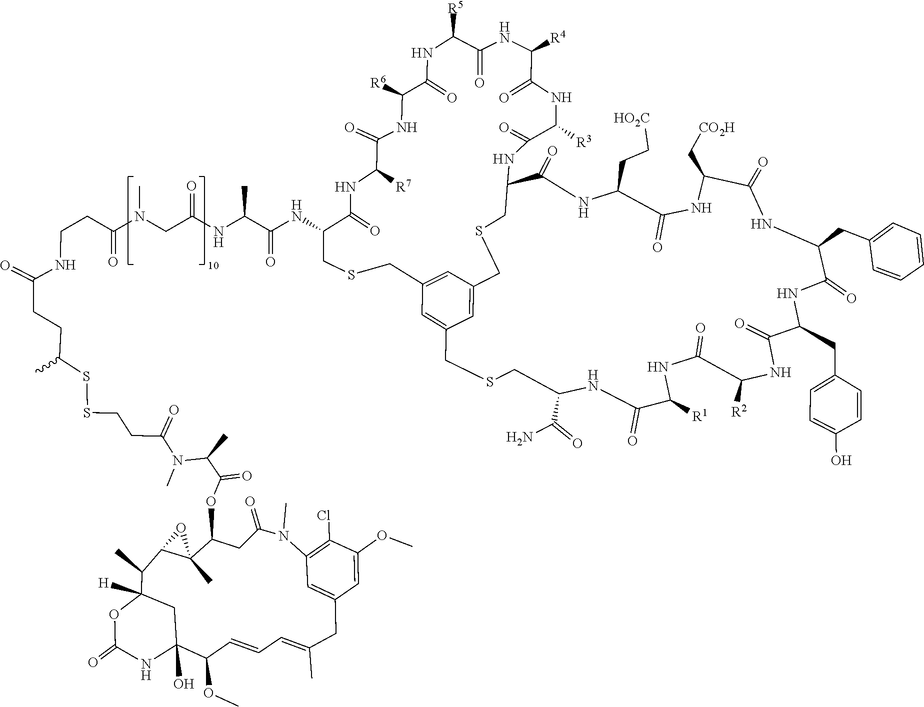

or a pharmaceutically acceptable salt thereof, wherein each of R¹, R², R³, R⁴, R⁵, R⁶, and R⁷ is as described in embodiments herein, both singly and in combination.

In some embodiments, a Bicycle toxin conjugate of formula (I) is BT1718, or a pharmaceutically acceptable salt thereof.

### 5. Uses, Formulation and Administration

Pharmaceutically Acceptable Compositions

According to another embodiment, the invention provides a composition comprising a Bicycle toxin conjugate of this invention, or a pharmaceutically acceptable derivative thereof, and a pharmaceutically acceptable carrier, adjuvant, or vehicle.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

In some embodiments, the present invention provides a method for preventing and/or treating cancers as described herein comprising administering to a patient a Bicycle toxin conjugate of the invention.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

### Cancer

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, a cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, a cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, a cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, a patient is an adult human. In some embodiments, a patient is a child or pediatric patient.

In some embodiments, a cancer includes, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, a cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, a cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is a solid tumor, such as a sarcoma, carcinoma, or lymphoma. Solid tumors generally comprise an abnormal mass of tissue that typically does not include cysts or liquid areas. In some embodiments, a cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, a cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, a cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is hepatocellular carcinoma (HCC). In some embodiments, a cancer is hepatoblastoma. In some embodiments, a cancer is colon cancer. In some embodiments, a cancer is rectal cancer. In some embodiments, a cancer is ovarian cancer, or ovarian carcinoma. In some embodiments, a cancer is ovarian epithelial cancer. In some embodiments, a cancer is fallopian tube cancer. In some embodiments, a cancer is papillary serous cystadenocarcinoma. In some embodiments, a cancer is uterine papillary serous carcinoma (UPSC). In some embodiments, a cancer is hepatocholangiocarcinoma. In some embodiments, a cancer is soft tissue and bone synovial sarcoma. In some embodiments, a cancer is rhabdomyosarcoma. In some embodiments, a cancer is osteosarcoma. In some embodiments, a cancer is anaplastic thyroid cancer. In some embodiments, a cancer is adrenocortical carcinoma. In some embodiments, a cancer is pancreatic cancer, or pancreatic ductal carcinoma. In some embodiments, a cancer is pancreatic adenocarcinoma. In some embodiments, the cancer is glioma. In some embodiments, a cancer is malignant peripheral nerve sheath tumors (MPNST). In some embodiments, a cancer is neurofibromatosis-1 associated MPNST. In some embodiments, a cancer is Waldenstrom's macroglobulinemia. In some embodiments, a cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, a cancer is melanoma cancer. In some embodiments, a cancer is breast cancer. In some embodiments, a cancer is lung cancer. In some embodiments, a cancer is small cell lung cancer (SCLC). In some embodiments, a cancer is non-small cell lung cancer (NSCLC).

In some embodiments, a cancer is treated by arresting further growth of the tumor. In some embodiments, a cancer is treated by reducing the size (e.g., volume or mass) of the tumor by at least 5%, 10%, 25%, 50%, 75%, 90% or 99% relative to the size of the tumor prior to treatment. In some embodiments, a cancer is treated by reducing the quantity of the tumor in the patient by at least 5%, 10%, 25%, 50%, 75%, 90% or 99% relative to the quantity of the tumor prior to treatment.

The compounds and compositions, according to the method of the present invention, may be administered using any amount and any route of administration effective for treating or lessening the severity of a cancer. The exact amount required will vary from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### EXEMPLIFICATION

The following Examples illustrate the invention described above; they are not, however, intended to limit the scope of the invention in any way. The beneficial effects of the pharmaceutical compounds, combinations, and compositions of the present invention can also be determined by other test models known as such to the person skilled in the pertinent art.

### List of Common Abbreviations Used in the Experimental Section

ACN: Acetonitrile
API: Active Pharmaceutical Ingredient
aq.: Aqueous
A %: Peak Area Percent
° C.: Degrees Celsius
CPP: Current Preferred Procedure
CV: Column Volumes
DMA: N,N-Dimethylacetamide
DM1: Mertansine/Emtansine
eq.: Molar Equivalents
h: Hours
TFA: Trifluoroacetic Acid
HPLC: High Performance Liquid Chromatography
USP: United States Pharmacopeia
IPC: In-process Control
mL: Milliliter
Mol: Moles
Mol. Wt.: Molecular Weight
MTBE: Methyl tert-Butyl Ether
non-GMP: Non-Good Manufacturing Practices
RP-18: Reverse Phase C18-bonded Silica
SPP: N-Succinimidyl 2-pyridyldithio-carboxylate
v/v: Volume/Volume
wt %: Weight Percent

### Example 1: Preparation of Bicycle Toxin Conjugate BT1718

The objective of this study is to develop a robust and scalable process for the manufacture of BT1718. The process is composed of two reactions and precipitations, followed by chromatographic purification and lyophilization.

The synthetic route consists of two steps: Step 1 amide formation of bicyclic peptide N241 and bifunctional linker SPP and step 2 disulfide exchange between N277 and DM1 (Scheme II).

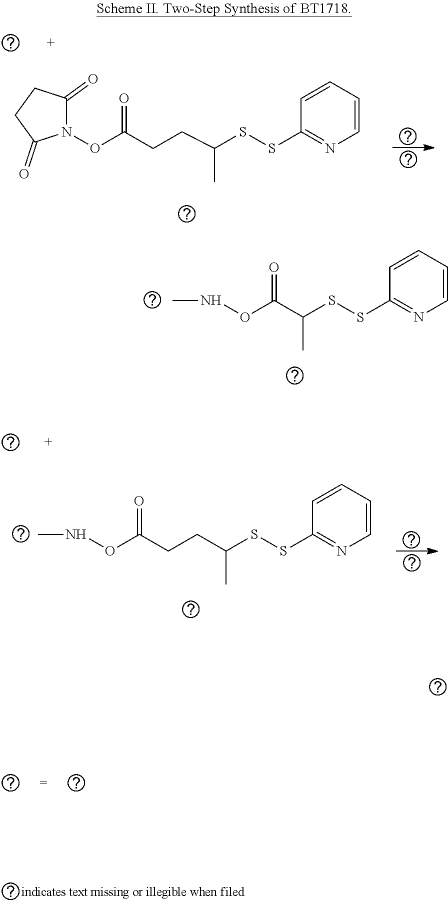 [SMILES: CC(CCC(=O)ON1C(=O)CCC1=O)SSc1ccccn1 ; CNOC(=O)C(C)SSc1ccccn1 ; CNOC(=O)CCC(C)SSc1ccccn1]

### Purification of N277 and BT1718

The reaction of N241 with SPP in DMA generated the coupling product N277 and N-hydroxysuccinimide as a side product. N277 reacted with DM1 to form the API BT1718 and a byproduct 2-pyridinethiol. Both N277 and BT1718 have good solubility in polar solvents like DMF and DMA but are not soluble in MTBE. Taking advantage of these properties, N277 and BT1718 were separated from impurities by precipitation with cold MTBE. As a result, N277 was isolated as a white powder with more than 94% purity, which was directly used for the second step. Disulfide exchange between N277 and DM1 gave BT1718. After the precipitation, BT1718 was obtained as a white solid with more than 82% purity. The crude BT1718 was further purified by RP-18 chromatography and lyophilized to give the API, which met the specifications.

### Results and Discussion

Scheme II consists of two step reactions used for the production of BT1718. In step 1, 1.3 eq. of SPP were utilized to achieve more than 99% conversion. Upon completion of the reaction, the reaction mixture was charged to cold MTBE (−20° C.) slowly. The resulting slurry was filtered and rinsed with MTBE to afford the intermediate N277. The side product N-hydroxysuccinimide and excess SPP were dissolved in the mother liquor. The isolated N277 was directly used for the second step without further purification. In step 2, a slight excess of DM1 (1.1 eq.) was utilized to achieve more than 98% conversion. Once the reaction reached completion, the reaction mixture was charged to cold MTBE (−20° C.) slowly. The resulting slurry was filtered and rinsed with MTBE to give the crude product BT1718 as a white solid. The crude BT1718 was dissolved in 25% acetonitrile in water. The solution was first purified by RP-18 column eluting with a mixed solvent of acetonitrile in water with 0.1% TFA from 10% to 45%. The eligible fractions were combined and concentrated under reduced pressure at room temperature to remove partial acetonitrile. The concentrate was then loaded onto the second RP-18 column eluting with 10% acetonitrile in water to remove TFA. The desired product was collected by eluting with 50% acetonitrile in water and lyophilized to give BT1718 as a white solid. Table 1 is a summary of process development of BT1718.

TABLE 1
Process Development of BT1718

| | | | | | N277 | D-DM1/ | D-BT1718/BT-1718 |
|---|---|---|---|---|---|---|---|
| Batch | N244 | | SPP | | (Yield/Purity) | DM1 | (Yield/Purity) |
| 1 | 430 | mg | 72 | mg | 410 mg | D-DM1 | D-BT1718 (70 mg) |
| | | | | | (88% yield/95 A %) | (34 mg) | (60 wt % yield over |
| | | | | | 110 mg for step 2 | | two steps/87 A %) |
| 2 | 430 | mg | 72 | mg | 410 mg | DM1 | BT1718 (90 mg) |
| | | | | | (88% yield/95 A %) | (48 mg) | (54 wt % yield over |
| | | | | | 177 mg for step 2 | | two steps/94 A %) |
| 3 | 1.06 | g | 177 | mg | 1.06 g | DM1 | BT1718 (650 mg) |
| | | | | | (91% yield/95 A %) | (298 mg) | (60 wt % yield over |
| | | | | | | | two steps/97 A %) |
| 4 | 1.6 | g | 226 | mg | 1.6 g | DM1 | BT1718 (980 mg, low |
| | | | | | (92% yield/95 A %) | (436 mg) | ACN/TFA content) |
| | | | | | | | (61 wt % yield over |
| | | | | | | | two steps/98 A %) |
| 5 | 50 | g | 8.3 | g | 51 g | 14.4 g | BT1718 (39 g, low |
| | | | | | (94% yield/94.5 A %) | (>95.0 A %) | ACN/TFA content) |
| | | | | | | | (>70 wt % yield over |
| | | | | | | | two steps/98.9 A %) |

### Step 1. Amide Formation of N241 and SPP

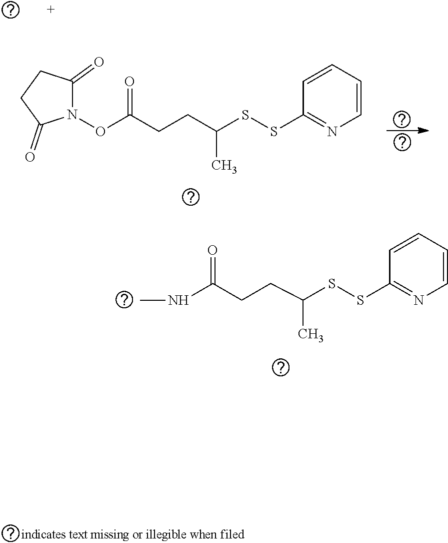 [SMILES: CC(CCC(=O)ON1C(=O)CCC1=O)SSc1ccccn1 ; CNC(=O)CCC(C)SSc1ccccn1]

A 1 L jacketed reactor equipped with a mechanical stirrer, thermocouple and nitrogen inlet was charged with a solution of N241 (50 g, 18.8 mmol, 1.0 eq.) in anhydrous DMA (300 mL) and a solution of SPP (8.31 g, 24.4 mmol, 1.3 eq.) in anhydrous DMA (300 mL). The resulting solution was charged with DIPEA (32.7 mL, 188 mmol, 10.0 eq.) over 5 min. The mixture was stirred at 22±2° C. for 3±0.5 h. HPLC analysis showed the complete reaction (>99.0% conversion; N241<1.0%).

The reaction mixture was transferred to a 1 L dropping funnel and slowly added to the pre-cooled MTBE (7.8 L, −20° C.) in a 10 L reactor. The 1 L dropping funnel was rinsed with 50 mL of DMA and the rinsate was charged to the reactor. The resulting slurry was stirred at −20° C. for 15 min and filtered through a class D filter. The cake was slurried with MTBE (1.3 L×3) and aspirated for a minimum of 30 min. The solid was dried at 22±2° C. for a minimum of 16 h until a constant weight (NMT 2% over 1 h). About 51 g of N277 was obtained as an off-white solid with 94% yield and 94.5 A % purity.

### Step 2. Disulfide Exchange of N277 and DM1

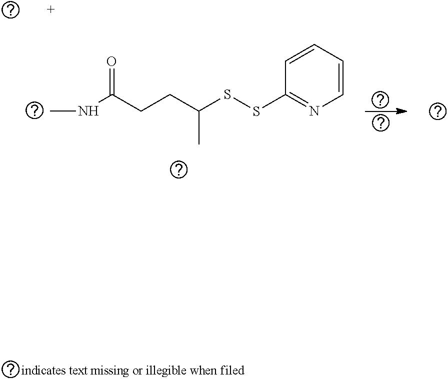 [SMILES: CNC(=O)CCC(C)SSc1ccccn1]

A 1 L jacketed reactor equipped with a mechanical stirrer, thermocouple and nitrogen inlet was charged with N277 (51 g, 17.7 mmol, 1.0 eq.) and anhydrous DMA (300 mL) at 22±2° C. The mixture was stirred until all solids were dissolved. A solution of DM1 (14.4 g, 19.5 mmol, 1.1 eq.) in anhydrous DMA (300 mL) was charged to the solution of N277 in DMA at 22° C. The resulting solution was charged with DIPEA (30.8 mL, 177 mmol, 10.0 eq.) over 5 min. The mixture was stirred at 22±2° C. for 3±0.5 h. HPLC analysis showed the complete reaction (>99.0% conversion; N277<1.0%).

The reaction mixture was transferred to a 1 L dropping funnel and slowly added to the pre-cooled MTBE (7.8 L, −20° C.) in a 10 L reactor. The 1 L dropping funnel was rinsed with 50 mL of DMA and the rinsate was charged to the reactor. The resulting slurry was stirred at −20° C. for 15 min and filtered through a class D filter. The cake was slurried with MTBE (1.3 L×3) and aspirated for a minimum of 30 min. The solid was dried at 22±2° C. for a minimum of 16 h until it passed a constant weight (NMT 2% over 1 h). The crude BT1718 (61 g) was split into three parts, which were purified by three Biotage RP-18 columns.

### Purification by RP-18 Column Chromatography

### Chromatography Parameters:

Stationary phase: Biotage RP-18 column
Column size: 1.85 kg

### Column Volume: 1 CV=2 L

Column equilibration (for new column): Used 5 CV of 90% (V/V) acetonitrile in water with 0.1% TFA then continued with column equilibration indicated below (Table 2).
Column equilibration: Used 5 CV of 10% (VN) acetonitrile in water with 0.1% TFA.
Mobile Phase: Prepared vessels and mobile phase as described in Table 2.
Loading of Crude BT1718 API Solution onto Column:

The crude BT1718 aqueous solution was loaded onto the Biotage RP-18 column and eluted with stepwise gradients. The solution was collected in ½ CV (1 L) fractions.

TABLE 2
Biotage C18 column (Column size = 1.85 kg, 1 CV = 2 L).

| | | Column | |
|---|---|---|---|
| Step | | Volumes (CV) | |
| gradient/ | Mobile phase | Note: 1 | |
| vessel ID | (V/V) | fraction = 1 L | Role |
| 1 | 90:10 water:ACN | 10 CV = 20 L | Elutes 2- |
| | with 0.1% TFA | | pyridinethiol |
| | | | (One fraction) |
| 2 | 80:20 water:ACN | 10 CV = 20 L | Elutes impurities |
| | with 0.1% TFA | | (One fraction) |
| 3 | 70:30 water:ACN | 10 CV = 20 L | Elutes impurities |
| | with 0.1% TFA | | (One fraction) |
| 4 | 65:35 water: ACN | 10 CV = 20 L | Elutes API |
| | with 0.1% TFA | | (BT1718) (Frs. |
| | | | 1-20) |
| 5 | 60:40 water:ACN | 10 CV = 20 L | Elutes API |
| | with 0.1% TFA | | (BT1718) (Frs. |
| | | | 21-40) |
| 6 | 55:45 water:ACN | 10 CV = 20 L | Elutes impurities |
| | with 0.1% TFA | | (Frs. 41-60) |

### Fractions Storage and Mapping

Fractions were stored at 2-8° C. during fractions analysis, and mapped by HPLC (Table 3) with more than 95.0% purity.

TABLE 3
HPLC Fraction Mapping (Fractions were collected from 30%)

| | Area % | Area |
|---|---|---|
| | BT1718 | (mAU*min) |
| Column A | | |
| Fraction 45 | 30.81 | 1.7014 |
| Fraction 46 | 64.03 | 6.6645 |
| Fraction 47 | 92.03 | 52.7922 |
| Fraction 48 | 98.87 | 237.1142 |
| Fraction 49 | 99.14 | 399.6134 |
| Fraction 50 | 96.84 | 399.5269 |
| Fraction 51 | 79.67 | 94.4248 |
| Fraction 52 | 23.58 | 11.1285 |
| Fraction 53 | 13.33 | 4.8679 |
| Column B | | |
| Fraction 24 | 2.31 | 0.1666 |
| Fraction 25 | 83.91 | 34.3008 |
| Fraction 26 | 98.07 | 249.2121 |
| Fraction 27 | 98.47 | 424.6574 |
| Fraction 28 | 97.25 | 449.5235 |
| Fraction 29 | 88.07 | 225.4126 |
| Fraction 30 | 37.11 | 25.0582 |
| Fraction 31 | 15.36 | 7.7522 |
| Fraction 34 | 38.25 | 1.8918 |
| Column C | | |
| Fraction 23 | 1.53 | 0.3285 |
| Fraction 24 | 52.46 | 6.0624 |
| Fraction 25 | 91.1 | 54.6196 |
| Fraction 26 | 98.78 | 293.5369 |
| Fraction 27 | 99.1 | 455.7847 |
| Fraction 28 | 96.32 | 437.4028 |
| Fraction 29 | 80.42 | 136.5746 |
| Fraction 30 | 26.8 | 16.503 |
| Fraction 31 | 13.83 | 6.2161 |
| Fraction 32 | 19.57 | 3.5495 |

Solvents Removal from Fractions

Fifteen fractions from the three columns were combined for HPLC analysis (96.9 A %). The composite was concentrated under reduced pressure on a Rotavap with its water bath set at 22° C. Upon formation of a suspension, the concentration stopped. A minimum of acetonitrile (10 mL) was charged to the suspension and a clear solution was formed.

### Removal of TFA by the Second RP-18 Column

The clear solution was loaded onto a 1.85 kg Biotage RP-18 column, which was pre-equilibrated with 10 CV of 90 v/v % ACN in water and 10 CV of 10 v/v % ACN in water, successively. The 20 L rotovap bulb was rinsed with 200 mL of 50% ACN in water. The column was first eluted with 20 CV of 10 v/v % ACN in water to remove TFA and then eluted with 10 CV of 50 v/v % ACN in water to collect 10 fractions. HPLC analysis showed that fractions 2-5 contained BT1718. The composite of the four fractions was analyzed by HPLC (98.3 A %).

### Lyophilization

The composite from the second column was filtered through a 0.45 uM PTFE syringe filter (30 mm diameter). The line was rinsed with 200 mL of 50% ACN in water. The filtrate was concentrated until a suspension appeared. A minimum amount of acetonitrile (10 mL) was charged to the suspension and a clear solution was formed. The solution was frozen in a freezer and the frozen cake was lyophilized for 3-4 days. After lyophilization, BT1718 was obtained as a white solid (39 g, 78 wt % yield). The resulting material was analyzed by HPLC (Purity 98.9 A %). CoA of the toxicological batch and MS spectrum are attached.

### CONCLUSION

A scalable process for the synthesis of BT1718 is developed with the following features:
The concentrations of the two steps were tripled, which gave a higher batch throughput (×3) of BT1718;
DMA was the only solvent utilized for the two reactions, which reduced analytical burden of testing for residual solvents;
The step 1 product N277 was isolated as a white solid by precipitation with cold MTBE (¬20° C.) instead of Akita FPLC separation;
The step 2 product BT1718 was isolated as a crude solid by precipitation with cold MTBE (−20° C.);
The crude product was purified by the first RP-18 column chromatography eluting with 10-45% ACN in water with 0.1% TFA. The new procedure gave very reproducible results (columns A, B and C), which shows potential to scale up to hundreds of grams. The old procedure utilized prep HPLC to purify the API, which has inherent limitation;
TFA in the combined fractions was readily purged by the second high-loading RP-18 plug eluting with 10% ACN in water. The product BT1718 was collected in high concentration when 50% ACN in water was used as an eluent;
The product solution was further concentrated and frozen in a freezer; and
The API BT1718 was obtained as a white fluffy solid after the lyophilization.

The improved process has been used to synthesize BT1718 from milligrams to tens of grams with consistent quality, and can potentially be used to manufacture BT1718 at hundreds of grams scale per batch.

We claim:

1. A method of preparing a compound of formula (I), or a salt thereof, comp rising steps of
1) providing compound B 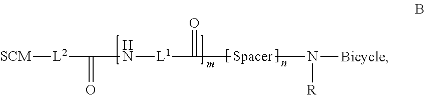
or a salt thereof;
2) reacting compound B with compound A 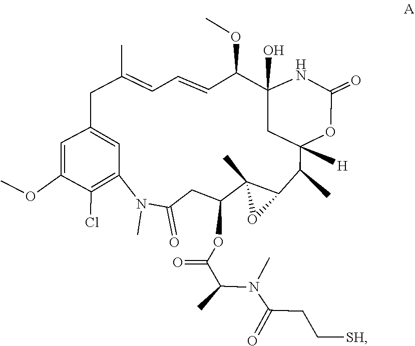 [SMILES: COc1cc2cc(c1Cl)N(C)C(=O)C[C@H](OC(=O)[C@H](C)N(C)C(=O)CCS)[C@@]1(C)O[C@H]1[C@H](C)[C@@H]1C[C@@](O)(NC(=O)O1)[C@H](OC)/C=C/C=C(\C)C2]
or a salt thereof, to form a compound of formula (I), 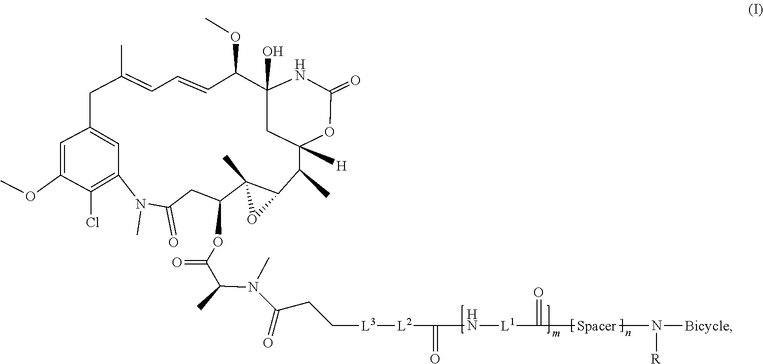
or a salt thereof; and
3) separating the compound of formula (I), or a salt thereof, from reaction mixture by precipitation in a non-polar solvent,
wherein:
Bicycle is a constrained bicyclic peptide that binds with high affinity and specificity to membrane type 1-matrix metalloprotease (MT1-MMP);
R is hydrogen or C₁₋₄ aliphatic;
Spacer is a natural or unnatural amino acid wherein the acid is connected to the N-terminus of Bicycle via an amide bond, or a peptide wherein the C-terminal acid of the peptide is connected to the N-terminus of Bicycle via an amide bond;
L¹ is a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—;
m is 0 or 1;
n is 0 or 1;
L² is a covalent bond or a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—;
each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, 10-12 membered saturated or partially unsaturated bicyclic heterocyclylene having 1-4 heteroatoms independently selected from nitrogen, oxygen, or sulfur, 10-12 membered saturated or partially unsaturated tricyclic heterocyclylene having 1-4 heteroatoms independently selected from nitrogen, oxygen, or sulfur, 10-12 membered partially saturated bicyclic heteroarylene having 1-4 heteroatoms independently selected from nitrogen, oxygen, or sulfur, 10-12 membered partially saturated tricyclic heteroarylene having 1-4 heteroatoms independently selected from nitrogen, oxygen, or sulfur, 9-12 membered bicyclic heteroarylene having 1-4 heteroatoms independently selected from nitrogen, oxygen, or sulfur, 19-20 membered partially unsaturated tetracyclic heteroarylene having 1-4 heteroatoms independently selected from nitrogen, oxygen, or sulfur, and 5-6 membered heteroarylene having 1-4 heteroatoms independently selected from nitrogen, oxygen, or sulfur. In some embodiments, each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, and 5-6 membered heteroarylene having 1-4 heteroatoms independently selected from nitrogen, oxygen, or sulfur;
SCM is a sulfhydryl crosslinking moiety that forms a bond with sulfhydryl (—SH); and
L³ is a group formed between —SH and SCM.

2. The method of claim 1, wherein step 2) reaction is in a dipolar aprotic solvent.

3. The method of claim 2, wherein the dipolar aprotic solvent is N,N-dimethylacetamide (DMA).

4. The method of any one of claims 1-3, wherein the non-polar solvent of step 3) is an ether.

5. The method of claim 4, wherein the ether is methyl tert-butyl ether (MTBE).

6. The method of any one of claims 1-5, further comprising purifying the compound of formula (I), or a salt thereof, by column chromatography.

7. A method of preparing compound B, or a salt thereof, comprising steps of
1) providing compound D 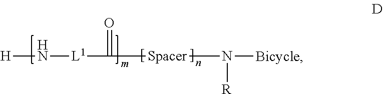
or a salt thereof;
2) reacting compound D with compound C 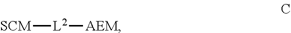
or a salt thereof, to form compound B 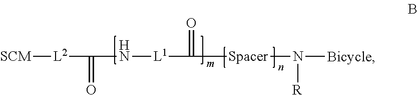
or a salt thereof; and
3) separating compound B, or a salt thereof, from reaction mixture by precipitation in a non-polar solvent,
wherein:
Bicycle is a constrained bicyclic peptide that binds with high affinity and specificity to membrane type 1-matrix metalloprotease (MT1-MMP);
R is hydrogen or C₁₋₄ aliphatic;
Spacer is a natural or unnatural amino acid wherein the acid is connected to the N-terminus of Bicycle via an amide bond, or a peptide wherein the C-terminal acid of the peptide is connected to the N-terminus of Bicycle via an amide bond;
L¹ is a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—;
m is 0 or 1;
n is 0 or 1;
AEM is 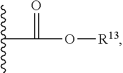
wherein —O—R¹³ is a leaving group;
L² is a covalent bond or a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—;
each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, 10-12 membered saturated or partially unsaturated bicyclic heterocyclylene having 1-4 heteroatoms independently selected from nitrogen, oxygen, or sulfur, 10-12 membered saturated or partially unsaturated tricyclic heterocyclylene having 1-4 heteroatoms independently selected from nitrogen, oxygen, or sulfur, 10-12 membered partially saturated bicyclic heteroarylene having 1-4 heteroatoms independently selected from nitrogen, oxygen, or sulfur, 10-12 membered partially saturated tricyclic heteroarylene having 1-4 heteroatoms independently selected from nitrogen, oxygen, or sulfur, 9-12 membered bicyclic heteroarylene having 1-4 heteroatoms independently selected from nitrogen, oxygen, or sulfur, 19-20 membered partially unsaturated tetracyclic heteroarylene having 1-4 heteroatoms independently selected from nitrogen, oxygen, or sulfur, and 5-6 membered heteroarylene having 1-4 heteroatoms independently selected from nitrogen, oxygen, or sulfur. In some embodiments, each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, and 5-6 membered heteroarylene having 1-4 heteroatoms independently selected from nitrogen, oxygen, or sulfur; and
SCM is a sulfhydryl crosslinking moiety that forms a bond with sulfhydryl (—SH).

8. The method of claim 7, wherein step 2) reaction is in a dipolar aprotic solvent.

9. The method of claim 8, wherein the dipolar aprotic solvent is N,N-dimethylacetamide (DMA).

10. The method of any one of claims 7-9, wherein the non-polar solvent of step 3) is an ether.

11. The method of claim 10, wherein the ether is methyl tert-butyl ether (MTBE).

12. The method of any one of claims 7-11, further comprising purifying compound B, or a salt thereof, by column chromatography.

13. A method of preparing a compound of formula (I), or a salt thereof, comprising steps of
1) providing compound D 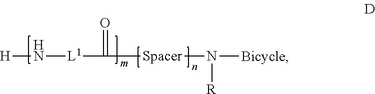
or a salt thereof;
2) reacting compound D with compound C 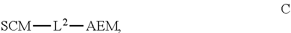
or a salt thereof, to form compound B 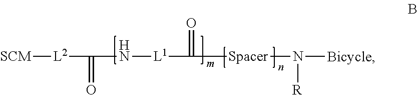
or a salt thereof;
3) separating compound B, or a salt thereof, from reaction mixture by precipitation in a non-polar solvent;
4) reacting compound B, or a salt thereof, with compound A 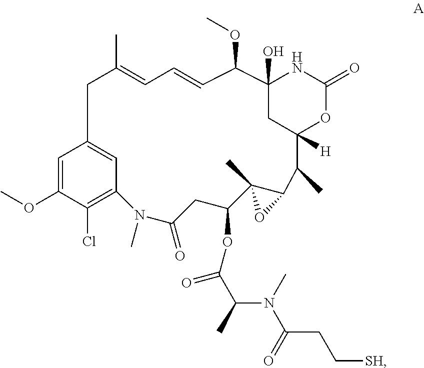 [SMILES: COc1cc2cc(c1Cl)N(C)C(=O)C[C@H](OC(=O)[C@H](C)N(C)C(=O)CCS)[C@@]1(C)O[C@H]1[C@H](C)[C@@H]1C[C@@](O)(NC(=O)O1)[C@H](OC)/C=C/C=C(\C)C2]
or a salt thereof, to form a compound of formula (I) 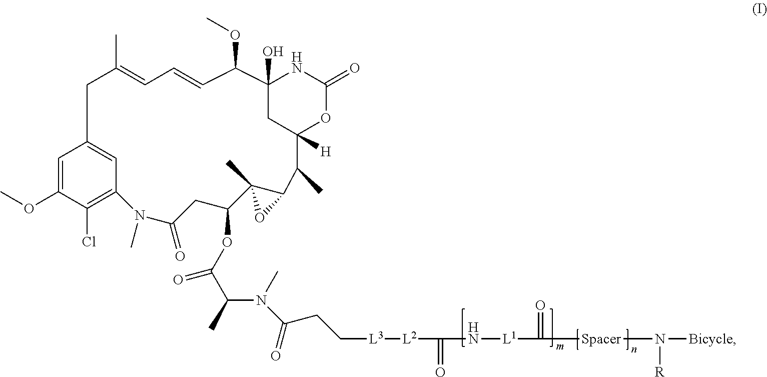
or a salt thereof; and
5) separating the compound of formula (I), or a salt thereof, from reaction mixture by precipitation in a non-polar solvent,
wherein:
Bicycle is a constrained bicyclic peptide that binds with high affinity and specificity to membrane type 1-matrix metalloprotease (MT1-MMP);
R is hydrogen or C₁₋₄ aliphatic;
Spacer is a natural or unnatural amino acid wherein the acid is connected to the N-terminus of Bicycle via an amide bond, or a peptide wherein the C-terminal acid of the peptide is connected to the N-terminus of Bicycle via an amide bond;
L¹ is a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—;
m is 0 or 1;
n is 0 or 1;
AEM is 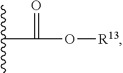
wherein —O—R¹³ is a leaving group;
L² is a covalent bond or a C₁₋₂₀ bivalent hydrocarbon chain wherein 1-5 methylene units of the chain are optionally and independently replaced by -Cy¹-, —S—, —N(R)—, —O—, —C(O)—, —OC(O)—, —C(O)O—, —C(O)N(R)—, —N(R)C(O)—, —OC(O)N(R)—, —N(R)C(O)O—, —S(O)—, —S(O)₂—, —C(CH₃)═N—N(R)—, —N(R)N═C(CH₃)—, —N(R)CH₂C(O)—, or —(CH₂CH₂O)₁₋₂₀—;
each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, 10-12 membered saturated or partially unsaturated bicyclic heterocyclylene having 1-4 heteroatoms independently selected from nitrogen, oxygen, or sulfur, 10-12 membered saturated or partially unsaturated tricyclic heterocyclylene having 1-4 heteroatoms independently selected from nitrogen, oxygen, or sulfur, 10-12 membered partially saturated bicyclic heteroarylene having 1-4 heteroatoms independently selected from nitrogen, oxygen, or sulfur, 10-12 membered partially saturated tricyclic heteroarylene having 1-4 heteroatoms independently selected from nitrogen, oxygen, or sulfur, 9-12 membered bicyclic heteroarylene having 1-4 heteroatoms independently selected from nitrogen, oxygen, or sulfur, 19-20 membered partially unsaturated tetracyclic heteroarylene having 1-4 heteroatoms independently selected from nitrogen, oxygen, or sulfur, and 5-6 membered heteroarylene having 1-4 heteroatoms independently selected from nitrogen, oxygen, or sulfur. In some embodiments, each -Cy¹- is independently an optionally substituted bivalent ring selected from phenylene, 3-7 membered saturated or partially unsaturated carbocyclylene, 4-7 membered saturated or partially unsaturated heterocyclylene having 1-2 heteroatoms independently selected from nitrogen, oxygen, or sulfur, and 5-6 membered heteroarylene having 1-4 heteroatoms independently selected from nitrogen, oxygen, or sulfur;
SCM is a sulfhydryl crosslinking moiety that forms a bond with sulfhydryl (—SH); and
L³ is a group formed between sulfhydryl (—SH) and SCM.

14. The method of claim 13, wherein step 2) reaction is in a dipolar aprotic solvent.

15. The method of claim 14, wherein the dipolar aprotic solvent is N,N-dimethylacetamide (DMA).

16. The method of any one of claims 13-15, wherein the non-polar solvent of step 3) is an ether.

17. The method of claim 16, wherein the ether is methyl tert-butyl ether (MTBE).

18. The method of any one of claims 13-17, wherein step 4) reaction is in a dipolar aprotic solvent.

19. The method of claim 18, wherein the dipolar aprotic solvent is N,N-dimethylacetamide (DMA).

20. The method of any one of claims 13-19, wherein the non-polar solvent of step 5) is an ether.

21. The method of claim 20, wherein the ether is methyl tert-butyl ether (MTBE).

22. The method of any one of claims 13-21, further comprising purifying compound B, or a salt thereof, by column chromatography before reacting to compound A, or a salt thereof, in step 4).

23. The method of any one of claims 13-22, further comprising purifying the compound of formula (I), or a salt thereof, by column chromatography.

24. The method of any one of the preceding claims, wherein Bicycle is: 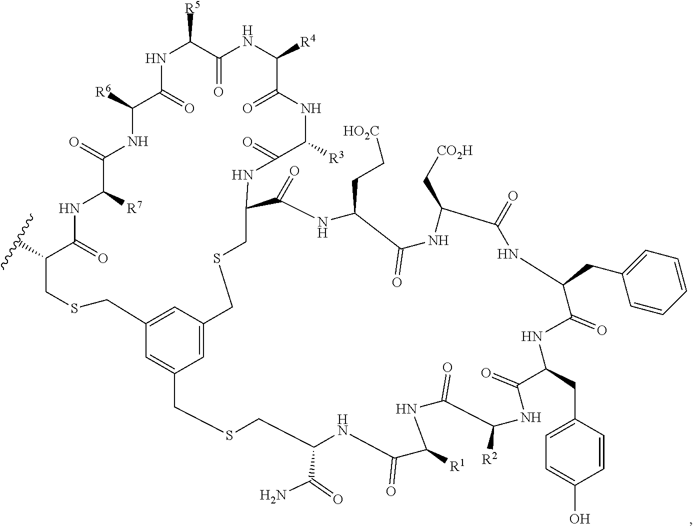
Wherein each of R¹, R², R³, R⁴, R⁵, R⁶, and R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

25. The method of claim 24, wherein Bicycle is:

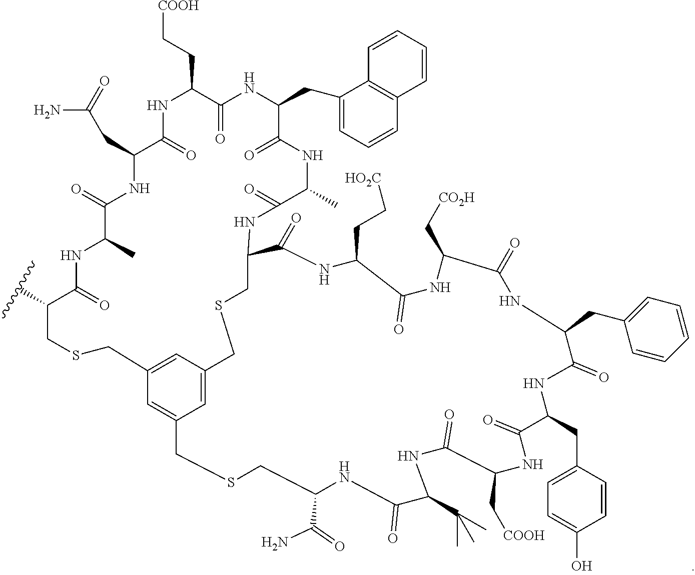 [SMILES: *[C@H]1CSCc2cc3cc(c2)CSC[C@H](NC(=O)[C@@H](C)NC(=O)[C@H](Cc2cccc4ccccc24)NC(=O)[C@H](CCC(=O)O)NC(=O)[C@H](CC(N)=O)NC(=O)[C@@H](C)NC1=O)C(=O)N[C@@H](CCC(=O)O)C(=O)N[C@@H](CC(=O)O)C(=O)N[C@@H](Cc1ccccc1)C(=O)N[C@@H](Cc1ccc(O)cc1)C(=O)N[C@@H](CC(=O)O)C(=O)N[C@@H](C(C)(C)C)C(=O)N[C@H](C(N)=O)CSC3]

26. The method of any one of the preceding claims, wherein R is hydrogen.

27. The method of any one of the preceding claims, wherein Spacer is

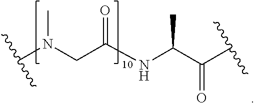

28. The method of any one of the preceding claims, wherein L¹ is —CH₂CH₂—.

29. The method of any one of the preceding claims, wherein L² is

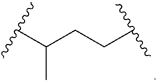 [SMILES: *CCC(*)C]

30. The method of any one of the preceding claims, wherein L³ is —S—S—.

31. The method of any one of the preceding claims, wherein compound D is 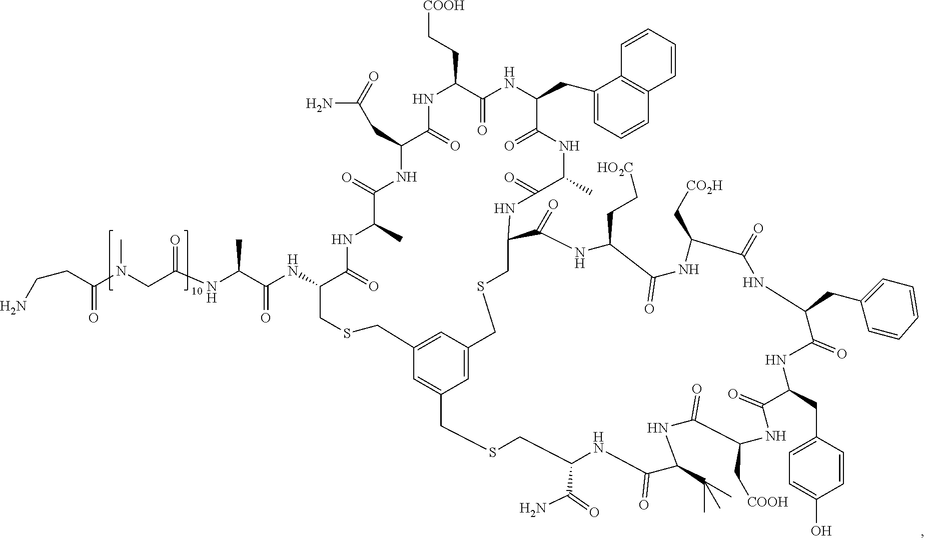
or a salt thereof.

32. The method of any one of the preceding claims, wherein compound C is 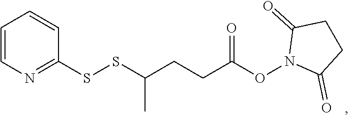 [SMILES: CC(CCC(=O)ON1C(=O)CCC1=O)SSc1ccccn1]
or a salt thereof.

33. The method of any one of the preceding claims, wherein compound B is 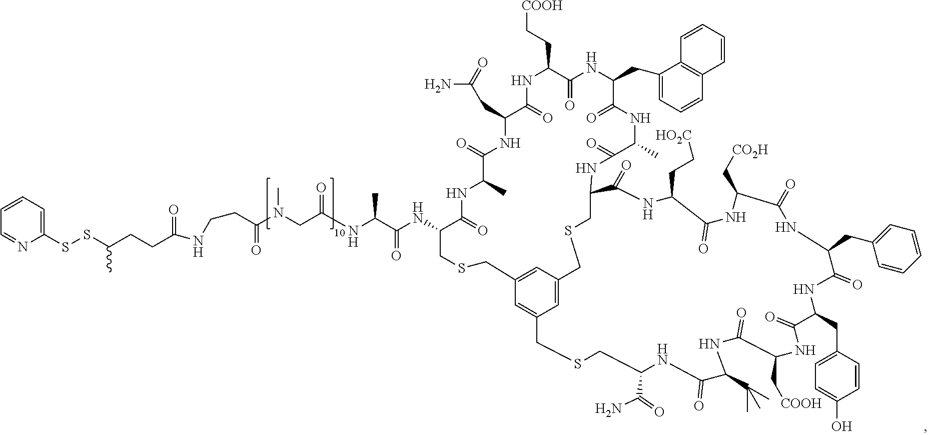
or a salt thereof.

34. The method of any one of the preceding claims, wherein the compound of formula (I) is BT1718, or a salt thereof.
